SEQUENCE LISTING (38 sequences)

SEQ ID NO: 1 - DNA, 30 bp, Artificial
  Synthetic
acgttggatgcctcttatttcagctgctgg

SEQ ID NO: 2 - DNA, 30 bp, Artificial
  Synthetic
acgttggatgagagaactctgattctggcg

SEQ ID NO: 3 - DNA, 30 bp, Artificial
  Synthetic
acgttggatgcaccaggaaccgtttatggc

SEQ ID NO: 4 - DNA, 30 bp, Artificial
  Synthetic
acgttggatgagcagctagaatcagaggag

SEQ ID NO: 5 - DNA, 30 bp, Artificial
  Synthetic
acgttggatgcctgccaaagaagaaacacc

SEQ ID NO: 6 - DNA, 30 bp, Artificial
  Synthetic
acgttggatgacgtctgcaggtatgtattc

SEQ ID NO: 7 - DNA, 30 bp, Artificial
  Synthetic
acgttggatggttcttaattcataggttgc

SEQ ID NO: 8 - DNA, 32 bp, Artificial
  Synthetic
acgttggatgcttcatttctcatcatattttc

SEQ ID NO: 9 - DNA, 30 bp, Artificial
  Synthetic
acgttggatgactgaagctccacaatttgg

SEQ ID NO: 10 - DNA, 31 bp, Artificial
  Synthetic
acgttggatggccactctagtactatatctg

SEQ ID NO: 11 - DNA, 30 bp, Artificial
  Synthetic
acgttggatgaggctgcaaaccagtggaac

SEQ ID NO: 12 - DNA, 30 bp, Artificial
  Synthetic
acgttggatgctgggcaaacaatgaaaatg

SEQ ID NO: 13 - DNA, 30 bp, Artificial
  Synthetic
acgttggatgtctagaaacacattggaagc

SEQ ID NO: 14 - DNA, 30 bp, Artificial
  Synthetic
acgttggatgcacggacatcattttccttc

SEQ ID NO: 15 - DNA, 30 bp, Artificial
  Synthetic
acgttggatgtcaaggactattgggagagc

SEQ ID NO: 16 - DNA, 30 bp, Artificial
  Synthetic
acgttggatgcagcagttgtacttgatgtc

SEQ ID NO: 17 - DNA, 26 bp, Artificial
  Synthetic
tactcctgcctctaggaaagaccaca

SEQ ID NO: 18 - DNA, 19 bp, Artificial
  Synthetic
gcaatctgctctatcctct

SEQ ID NO: 19 - DNA, 22 bp, Artificial
  Synthetic
tacttatttacgcttgaacctc

SEQ ID NO: 20 - DNA, 23 bp, Artificial
  Synthetic
cttaattcataggttgcaatttt

SEQ ID NO: 21 - DNA, 21 bp, Artificial
  Synthetic
cacaatttggtgaattatcaa

SEQ ID NO: 22 - DNA, 25 bp, Artificial
  Synthetic
aacccacagagctgctttgtatttc

SEQ ID NO: 23 - DNA, 17 bp, Artificial
  Synthetic
cattggaagctgcgctc

SEQ ID NO: 24 - DNA, 19 bp, Artificial
  Synthetic
tcatcaaagccccaggcta

SEQ ID NO: 25 - DNA, 27 bp, Artificial
  Synthetic
tactcctgcctctaggaaagaccacac

SEQ ID NO: 26 - DNA, 20 bp, Artificial
  Synthetic
gcaatctgctctatcctctt

SEQ ID NO: 27 - DNA, 23 bp, Artificial
  Synthetic
tacttatttacgcttgaacctca

SEQ ID NO: 28 - DNA, 24 bp, Artificial
  Synthetic
cttaattcataggttgcaatttta

SEQ ID NO: 29 - DNA, 22 bp, Artificial
  Synthetic
cacaatttggtgaattatcaat

SEQ ID NO: 30 - DNA, 26 bp, Artificial
  Synthetic
aacccacagagctgctttgtatttca

SEQ ID NO: 31 - DNA, 27 bp, Artificial
  Synthetic
tactcctgcctctaggaaagaccacat

SEQ ID NO: 32 - DNA, 21 bp, Artificial
  Synthetic
gcaatctgctctatcctctgc

SEQ ID NO: 33 - DNA, 24 bp, Artificial
  Synthetic
tacttatttacgcttgaacctcga

SEQ ID NO: 34 - DNA, 25 bp, Artificial
  Synthetic
cttaattcataggttgcaattttgt

SEQ ID NO: 35 - DNA, 23 bp, Artificial
  Synthetic
cacaatttggtgaattatcaaat

SEQ ID NO: 36 - DNA, 26 bp, Artificial
  Synthetic
aacccacagagctgctttgtatttcg

SEQ ID NO: 37 - DNA, 51 bp, Artificial
  Synthetic
agacgacactagtttttaaagttttkattaatcgctgctgtgaagctgcat

SEQ ID NO: 38 - DNA, 51 bp, Artificial
  Synthetic
gaaattgttttctttggacaacttgrcaaaaaccaatcatctgtcagtgat

(19) United States
(12) Patent Application Publication
(10) Pub. No.: US 2016/0076104 A1
(43) Pub. Date: Mar. 17, 2016

(54) METHODS AND COMPOSITIONS FOR ASSESSMENT OF PULMONARY FUNCTION AND DISORDERS

(71) Applicant: Robert Peter Young, Auckland (NZ)

(72) Inventor: Robert Peter Young, Auckland (NZ)

(73) Assignee: SYNERGENZ BIOSCIENCE LTD, HONG KONG (CN)

(21) Appl. No.: 14/949,607

(22) Filed: Nov. 23, 2015

Related U.S. Application Data
(63) Continuation of application No. 14/027,075, filed on Sep. 13, 2013, now abandoned.
Application No. 14/027,075 is a continuation of application No. 12/992,596, filed on Mar. 9, 2011, now abandoned.

(30) Foreign Application Priority Data
May 12, 2008 (NZ) 568247

(51) Int. Cl. C12Q 1/68

(52) U.S. Cl. CPC C12Q 1/6883; C12Q 2600/156; A61K 48/00

(57) ABSTRACT

The present invention provides methods for the assessment of risk of developing chronic obstructive pulmonary disease (COPD), emphysema or both COPD and emphysema in smokers and non-smokers using analysis of genetic polymorphisms.

### FIELD OF THE INVENTION

The present invention is concerned with methods for assessment of pulmonary function and/or disorders, and in particular for assessing risk of developing chronic obstructive pulmonary disease (COPD) and emphysema in smokers and non-smokers using analysis of genetic polymorphisms and altered gene expression. The present invention is also concerned with the use of genetic polymorphisms in the assessment of a subject's risk of developing COPD and emphysema.

### BACKGROUND OF THE INVENTION

Chronic obstructive pulmonary disease (COPD) is the 4^{th} leading cause of death in developed countries and a major cause for hospital readmission world-wide. It is characterised by insidious inflammation and progressive lung destruction. It becomes clinically evident after exertional breathlessness is noted by affected smokers when 50% or more of lung function has already been irreversibly lost. This loss of lung function is detected clinically by reduced expiratory flow rates (specifically forced expiratory volume in one second or FEV1). Over 95% of COPD is attributed to cigarette smoking yet only 20% or so of smokers develop COPD (susceptible smoker). Studies surprisingly show that smoking dose accounts for only about 16% of the impaired lung function. A number of family studies comparing concordance in siblings (twins and non-twin) consistently show a strong familial tendency and the search for COPD disease-susceptibility (or disease modifying) genes is underway.

Despite advances in the treatment of airways disease, current therapies do not significantly alter the natural history of COPD with progressive loss of lung function causing respiratory failure and death. Although cessation of smoking has been shown to reduce this decline in lung function if this is not achieved within the first 20 years or so of smoking for susceptible smokers, the loss is considerable and symptoms of worsening breathlessness cannot be averted. Smoking cessation studies indicate that techniques to help smokers quit have limited success. Analogous to the discovery of serum cholesterol and its link to coronary artery disease, there is a need to better understand the factors that contribute to COPD so that tests that identify at risk smokers can be developed and that new treatments can be discovered to reduce the adverse effects of smoking.

A number of epidemiology studies have consistently shown that at exposure doses of 20 or more pack years, the distribution in lung function tends toward trimodality with a proportion of smokers maintaining normal lung function (resistant smokers) even after 60+ pack years, a proportion showing modest reductions in lung function who may never develop symptoms and a proportion who show an accelerated loss in lung function who invariably develop COPD. This suggests that amongst smokers 3 populations exist, those resistant to developing COPD, those at modest risk and those at higher risk (termed susceptible smokers).

COPD is a heterogeneous disease encompassing, to varying degrees, emphysema and chronic bronchitis which develop as part of a remodelling process following the inflammatory insult from chronic tobacco smoke exposure and other air pollutants. It is likely that many genes are involved in the development of COPD.

To date, a number of biomarkers useful in the diagnosis and assessment of propensity towards developing various pulmonary disorders have been identified. These include, for example, single nucleotide polymorphisms including the following: A-82G in the promoter of the gene encoding human macrophage elastase (MMP12); T→C within codon 10 of the gene encoding transforming growth factor beta (TGFβ); C+760G of the gene encoding superoxide dismutase 3 (SOD3); T-1296C within the promoter of the gene encoding tissue inhibitor of metalloproteinase 3 (TIMP3); and polymorphisms in linkage disequilibrium (LD) with these polymorphisms, as disclosed in PCT International Application PCT/NZ02/00106 (published as WO 02/099134 and incorporated herein in its entirety).

It would be desirable and advantageous to have additional biomarkers which could be used to assess a subject's risk of developing pulmonary disorders such as chronic obstructive pulmonary disease (COPD) and emphysema, or a risk of developing COPD/emphysema-related impaired lung function, particularly if the subject is a smoker, and/or to provide the public with a useful choice.

It is primarily to such biomarkers and their use in methods to assess risk of developing such disorders that the present invention is directed.

### SUMMARY OF THE INVENTION

The present invention is primarily based on the finding that certain polymorphisms are found more often in subjects with COPD, emphysema, or both COPD and emphysema than in control subjects. Analysis of these polymorphisms reveals an association between genotypes and the subject's risk of developing COPD, emphysema, or both COPD and emphysema.

Thus, according to one aspect there is provided a method of determining a subject's risk of developing one or more obstructive lung diseases comprising analysing a sample from said subject for the presence or absence of one or more polymorphisms selected from the group comprising, consisting essentially of, or consisting of:
rs10115703 G/A polymorphism in the gene encoding Cerberus 1 (Cer 1);
rs13181 G/T polymorphism in the gene encoding xeroderma pigmentosum complementation group D (XPD);
rs1799930 G/A polymorphism in the gene encoding N-Acetyl transferase 2 (NAT2);
rs2031920 C/T polymorphism in the gene encoding cytochrome P450 2E1 (CYP2E1);
rs4073 T/A polymorphism in the gene encoding Interleukin8 (IL-8);
rs763110 C/T polymorphism in the gene encoding Fas ligand (FasL);
rs16969968 G/A polymorphism in the gene encoding α5 nicotinic acetylcholine receptor subunit (α5-nAChR); or
rs1051730 C/T polymorphism in the gene encoding α5-nAChR;

wherein the presence or absence of one or more of said polymorphisms is indicative of the subject's risk of developing one or more obstructive lung diseases selected from the group consisting of chronic obstructive pulmonary disease (COPD), emphysema, or both COPD and emphysema.

The one or more polymorphisms can be detected directly or by detection of one or more polymorphisms which are in linkage disequilibrium with said one or more polymorphisms.

Linkage disequilibrium (LD) is a phenomenon in genetics whereby two or more mutations or polymorphisms are in such close genetic proximity that they are co-inherited. This means that in genotyping, detection of one polymorphism as present infers the presence of the other. (Reich D E et al; Linkage disequilibrium in the human genome, Nature 2001, 411:199-204.)

The method can additionally comprise analysing a sample from said subject for the presence of one or more further polymorphisms selected from the group comprising, consisting essentially of, or consisting of:
the rs4934 G/A polymorphism in the gene encoding α1 anti-chymotrypsin;
the rs1489759 A/G polymorphism in the gene encoding Hedgehog interacting protein (HHIP);
the rs2202507 A/C polymorphism in the gene encoding Glycophorin A (GYPA).

The method can additionally comprise analysing a sample from said subject for the presence of one or more further polymorphisms selected from the group comprising, consisting essentially of, or consisting of:
−765 C/G in the promoter of the gene encoding Cyclooxygenase 2 (COX2);
105 C/A in the gene encoding Interleukinl8 (IL18);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tumour Necrosis Factor α (TNFα);
C89Y A/G in the gene encoding SMAD3;
E 469 K A/G in the gene encoding Intracellular Adhesion molecule 1 (ICAM1);
Gly 881Arg G/C in the gene encoding Caspase (NOD2);
161 G/A in the gene encoding Mannose binding lectin 2 (MBL2);
−1903 G/A in the gene encoding Chymase 1 (CMA1);
Arg 197 Gln G/A in the gene encoding N-Acetyl transferase 2 (NAT2);
−366 G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1 +49 C/T in the gene encoding Elafin; or
−1607 1G/2G in the promoter of the gene encoding Matrix Metalloproteinase 1 (MMP1), with reference to the 1G allele only;
16Arg/Gly in the gene encoding β2 Adrenergic Receptor (ADBR);
130 Arg/Gln (G/A) in the gene encoding Interleukin13 (IL13);
298 Asp/Glu (T/G) in the gene encoding Nitric oxide Synthase 3 (NO53);
Ile 105 Val (A/G) in the gene encoding Glutathione S Transferase P (GST-P);
Glu 416 Asp (T/G) in the gene encoding Vitamin D binding protein (VDBP);
Lys 420 Thr (A/C) in the gene encoding VDBP;
−1055 C/T in the promoter of the gene encoding IL13;
−308 G/A in the promoter of the gene encoding TNFα;
−511 A/G in the promoter of the gene encoding Interleukin 1B (IL1B);
Tyr 113 His T/C in the gene encoding Microsomal epoxide hydrolase (MEH);
His139 Arg G/A in the gene encoding MEH;
Gln 27 Glu C/G in the gene encoding ADBR;
−1607 1G/2G in the promoter of the gene encoding Matrix Metalloproteinase 1 (MMP1) with reference to the 2G allele only;
−1562 C/T in the promoter of the gene encoding Metalloproteinase 9 (MMP9);
M1 (GSTM1) null in the gene encoding Glutathione S Transferase 1 (GST-1);
1237 G/A in the 3′ region of the gene encoding α1-antitrypsin;
−82 A/G in the promoter of the gene encoding MMP12;
T→C within codon 10 of the gene encoding TGFβ;
760 C/G in the gene encoding SOD3;
−1296 T/C within the promoter of the gene encoding TIMP3; or
the S mutation in the gene encoding α1-antitrypsin.

Again, detection of the one or more further polymorphisms may be carried out directly or by detection of polymorphisms in linkage disequilibrium with the one or more further polymorphisms.

The presence of one or more polymorphisms selected from the group consisting of:
the G allele at the rs13181 polymorphism in the gene encoding XPD;
the GG genotype at the rs13181 polymorphism in the gene encoding XPD;
the T allele at the rs763110 polymorphism in the gene encoding FasL; or
the TT genotype at the rs763110 polymorphism in the gene encoding FasL;
the G allele at the rs1489759 polymorphism in the gene encoding HHIP;
the GG genotype at the rs 1489759 polymorphism in the gene encoding HHIP;
the C allele at the rs2202507 polymorphism in the gene encoding GYPA;
the CC genotype at the rs2202507 polymorphism in the gene encoding GYPA;
   may be indicative of a reduced risk of developing COPD, emphysema, or both COPD and emphysema.

The presence of one or more polymorphisms selected from the group consisting of:
the A allele at the rs10115703 polymorphism in the gene encoding Cer 1;
the GA genotype or AA genotype at the rs10115703 polymorphism in the gene encoding Cer 1;
the G allele at the rs1799930 polymorphism in the gene encoding NAT2;
the GG genotype at the rs1799930 polymorphism in the gene encoding NAT2;
the T allele at the rs2031920 polymorphism in the gene encoding CYP2E1;
the CT genotype or TT genotype at the rs2031920 polymorphism in the gene encoding CYP2E1;
the T allele at the rs4073 polymorphism in the gene encoding IL-8;
the TT genotype at the rs4073 polymorphism in the gene encoding IL-8;
the A allele at the rs16969968 polymorphism in the gene encoding α5-nAChR;
the AA genotype at the rs16969968 polymorphism in the gene encoding α5-nAChR;
the T allele at the rs1051730 polymorphism in the gene encoding α5-nAChR;
the TT genotype at the rs1051730 polymorphism in the gene encoding α5-nAChR;
the G allele at the rs4934 polymorphism in the gene encoding α1 anti-chymotrypsin; or
the GG genotype at the rs4934 polymorphism in the gene encoding α1 anti-chymotrypsin; may be indicative of an increased risk of developing COPD, emphysema, or both COPD and emphysema.

The methods of the invention are particularly useful in smokers (both current and former).

It will be appreciated that the methods of the invention identify two categories of polymorphisms—namely those associated with a reduced risk of developing COPD, emphysema, or both COPD and emphysema (which can be termed “protective polymorphisms”) and those associated with an increased risk of developing COPD, emphysema, or both COPD and emphysema (which can be termed “susceptibility polymorphisms”).

Therefore, the present invention further provides a method of assessing a subject's risk of developing chronic obstructive pulmonary disease (COPD), emphysema, or both COPD and emphysema, said method comprising providing the result of one or more genetic tests of a sample from the subject, and analysing the result for the presence or absence of one or more polymorphisms selected from the group comprising, consisting essentially of, or consisting of:
rs10115703 G/A polymorphism in the gene encoding Cer 1;
rs13181 G/T polymorphism in the gene encoding XPD;
rs1799930 G/A polymorphism in the gene encoding NAT2;
rs2031920 C/T polymorphism in the gene encoding CYP2E1;
rs4073 T/A polymorphism in the gene encoding IL-8;
rs763110 C/T polymorphism in the gene encoding FasL;
rs16969968 G/A polymorphism in the gene encoding α5-nAChR;
rs1051730 C/T polymorphism in the gene encoding α5 nicotinic acetylcholine receptor subunit (α5-nAChR);

wherein the presence or absence of one or more of said polymorphisms is indicative of the subject's risk of developing COPD, emphysema, or both COPD and emphysema.

The method can additionally comprise analysing the result for the presence of one or more further polymorphisms selected from the group comprising, consisting essentially of, or consisting of:
the rs4934 G/A polymorphism in the gene encoding α1 anti-chymotrypsin;
the rs1489759 A/G polymorphism in the gene encoding Hedgehog interacting protein (HHIP); or
the rs2202507 A/C polymorphism in the gene encoding Glycophorin A (GYPA).

The method can additionally comprise analysing the result for the presence of one or more further polymorphisms described above.

In a preferred form of the invention the presence of two or more protective polymorphisms is indicative of a reduced risk of developing COPD, emphysema, or both COPD and emphysema.

In a further preferred form of the invention the presence of two or more susceptibility polymorphisms is indicative of an increased risk of developing COPD, emphysema, or both COPD and emphysema.

In still a further preferred form of the invention the presence of two or more protective polymorphims irrespective of the presence of one or more susceptibility polymorphisms is indicative of reduced risk of developing COPD, emphysema, or both COPD and emphysema.

In one particularly preferred form of the invention there is provided a method of determining a subject's risk of developing chronic obstructive pulmonary disease (COPD), emphysema, or both COPD and emphysema, the method comprising providing the result of one or more genetic tests of a sample from the subject, and analysing the result for the presence or absence of two or more, three or more, four or more, five or more, six or more, seven or more, eight or more, or nine of the polymorphisms selected from the group consisting of:
rs10115703 G/A polymorphism in the gene encoding Cer 1;
rs13181 G/T polymorphism in the gene encoding XPD;
rs1799930 G/A polymorphism in the gene encoding NAT2;
rs2031920 C/T polymorphism in the gene encoding CYP2E1;
rs4073 T/A polymorphism in the gene encoding IL-8;
rs763110 C/T polymorphism in the gene encoding FasL;
rs16969968 G/A polymorphism in the gene encoding α5-nAChR;
rs1051730 C/T polymorphism in the gene encoding α5-nAChR; or
rs4934 G/A polymorphism in the gene encoding α1 anti-chymotrypsin;

wherein the presence or absence of two or more of said polymorphisms is indicative of the subject's risk of developing COPD, emphysema, or both COPD and emphysema.

The method can additionally comprise analysing a sample from said subject for the presence or absence of one or more further polymorphisms described above.

In a preferred form of the invention the methods as described herein are performed in conjunction with an analysis of one or more risk factors, including one or more epidemiological risk factors, associated with a risk of developing chronic obstructive pulmonary disease (COPD) and/or emphysema. Such epidemiological risk factors include but are not limited to smoking or exposure to tobacco smoke, age, sex, and familial history of COPD, emphysema, or both COPD and emphysema.

In another aspect the invention provides a set of nucleotide probes and/or primers for use in the preferred methods of the invention herein described. Preferably, the nucleotide probes and/or primers are those which span, or are able to be used to span, the polymorphic regions of the genes.

In one embodiment, the set of nucleotide probes and/or primers includes one or more primers or primer pairs which span or are able to be used to span one or more of the polymorphisms selected from the group comprising, consisting essentially of, or consisting of:
the rs10115703 G/A polymorphism in the gene encoding Cer 1;
the rs13181 G/T polymorphism in the gene encoding XPD;
the rs1799930 G/A polymorphism in the gene encoding NAT2;
the rs2031920 C/T polymorphism in the gene encoding CYP2E1;
the rs4073 T/A polymorphism in the gene encoding IL-8;
the rs763110 C/T polymorphism in the gene encoding FasL;
the rs16969968 G/A polymorphism in the gene encoding α5-nAChR; or
the rs1051730 C/T polymorphism in the gene encoding α5-nAChR.

In one example, one or more primers or primer pairs are included for one or more, two or more, three or more, four or more, five or more, six or more, seven or more, eight or more, or nine of the above polymorphisms.

In a further embodiment, the set of nucleotide probes and/or primers includes one or more primers or primer pairs for one or more of the further polymorphisms described above.

Also provided are one or more nucleotide probes and/or primers comprising the sequence of any one of the probes and/or primers herein described, including any one comprising or consisting of the sequence of any one of SEQ. ID. NO. 1 to 38, more preferably any one of SEQ. ID. NO. 1 to 24.

In yet a further aspect, the invention provides a nucleic acid microarray for use in the methods of the invention, which microarray comprises a substrate presenting nucleic acid sequences capable of hybridizing to nucleic acid sequences which encode one or more of the susceptibility or protective polymorphisms described herein or sequences complimentary thereto.

In one embodiment, the presence or absence of one or more of the above alleles or genotypes is determined with respect to a polynucleotide (genomic DNA, mRNA or cDNA produced from mRNA) comprising the polymorphism obtained from the subject.

In one embodiment, the presence or absence of one or more of the above alleles or genotypes is determined by sequencing the polynucleotide obtained from the subject.

In a further embodiment the determination comprises the step of amplifying a polynucleotide sequence from genomic DNA, mRNA or cDNA produced from mRNA comprising the polymorphism derived from said mammalian subject, for example by PCR.

Preferably the determination is by use of primers which comprise a nucleotide sequence having at least about 12 contiguous bases of or complementary to a sequence comprising the polymorphism or a naturally occurring flanking sequence.

In yet a further aspect, the invention provides a nucleic acid microarray for use in the methods of the invention, which microarray comprises a substrate presenting nucleic acid sequences capable of hybridizing to nucleic acid sequences which encode one or more of the susceptibility or protective polymorphisms described herein or sequences complimentary thereto.

In another aspect, the invention provides an antibody microarray for use in the methods of the invention, which microarray comprises a substrate presenting antibodies capable of binding to a product of expression of a gene the expression of which is upregulated or downregulated when associated with a susceptibility or protective polymorphism as described herein.

In a further aspect the present invention provides a method of treating a subject having an increased risk of developing COPD, emphysema, or both COPD and emphysema comprising the step of replicating, genotypically or phenotypically, the presence and/or functional effect of a protective polymorphism in said subject.

In yet a further aspect, the present invention provides a method of treating a subject having an increased risk of developing COPD, emphysema, or both COPD and emphysema, said subject having a detectable susceptibility polymorphism which either upregulates or downregulates expression of a gene such that the physiologically active concentration of the expressed gene product is outside a range which is normal for the age and sex of the subject, said method comprising the step of restoring the physiologically active concentration of said product of gene expression to be within a range which is normal for the age and sex of the subject.

In yet a further aspect, the present invention provides a method for screening for compounds that modulate the expression and/or activity of a gene, the expression of which is upregulated or downregulated when associated with a susceptibility or protective polymorphism, said method comprising the steps of:

contacting a candidate compound with a cell comprising a susceptibility or protective polymorphism which has been determined to be associated with the upregulation or downregulation of expression of a gene; and

measuring the expression of said gene following contact with said candidate compound,

wherein a change in the level of expression after the contacting step as compared to before the contacting step is indicative of the ability of the compound to modulate the expression and/or activity of said gene.

Preferably, said cell is a human lung cell which has been pre-screened to confirm the presence of said polymorphism.

Preferably, said cell comprises a susceptibility polymorphism associated with upregulation of expression of said gene and said screening is for candidate compounds which downregulate expression of said gene.

Alternatively, said cell comprises a susceptibility polymorphism associated with downregulation of expression of said gene and said screening is for candidate compounds which upregulate expression of said gene.

In another embodiment, said cell comprises a protective polymorphism associated with upregulation of expression of said gene and said screening is for candidate compounds which further upregulate expression of said gene.

Alternatively, said cell comprises a protective polymorphism associated with downregulation of expression of said gene and said screening is for candidate compounds which further downregulate expression of said gene.

In another aspect, the present invention provides a method for screening for compounds that modulate the expression and/or activity of a gene, the expression of which is upregulated or downregulated when associated with a susceptibility or protective polymorphism, said method comprising the steps of:

contacting a candidate compound with a cell comprising a gene, the expression of which is upregulated or downregulated when associated with a susceptibility or protective polymorphism but which in said cell the expression of which is neither upregulated nor downregulated; and

measuring the expression of said gene following contact with said candidate compound, wherein a change in the level of expression after the contacting step as compared to before the contacting step is indicative of the ability of the compound to modulate the expression and/or activity of said gene.

Preferably, said cell is human lung cell which has been pre-screened to confirm the presence, and baseline level of expression, of said gene.

Preferably, expression of the gene is downregulated when associated with a susceptibility polymorphism and said screening is for candidate compounds which in said cell, upregulate expression of said gene.

Alternatively, expression of the gene is upregulated when associated with a susceptibility polymorphism and said screening is for candidate compounds which, in said cell, downregulate expression of said gene.

In another embodiment, expression of the gene is upregulated when associated with a protective polymorphism and said screening is for compounds which, in said cell, upregulate expression of said gene.

Alternatively, expression of the gene is downregulated when associated with a protective polymorphism and said screening is for compounds which, in said cell, downregulate expression of said gene.

In yet a further aspect, the present invention provides a method of assessing the likely responsiveness of a subject at risk of developing or suffering from COPD, emphysema, or both COPD and emphysema to a prophylactic or therapeutic treatment, which treatment involves restoring the physiologically active concentration of a product of gene expression to be within a range which is normal for the age and sex of the subject, which method comprises detecting in said subject the presence or absence of a susceptibility polymorphism which when present either upregulates or downregulates expression of said gene such that the physiological active concentration of the expressed gene product is outside said normal range, wherein the detection of the presence of said polymorphism is indicative of the subject likely responding to said treatment.

In a further aspect, the present invention provides a kit for assessing a subject's risk of developing one or more obstructive lung diseases selected from COPD, emphysema, or both COPD and emphysema, said kit comprising a means of analysing a sample from said subject for the presence or absence of one or more polymorphisms disclosed herein.

In other aspects, the invention provides a system for performing one or more of the methods of the invention, said system comprising:

computer processor means for receiving, processing and communicating data;

storage means for storing data including a reference genetic database of the results of genetic analysis of a mammalian subject with respect to predisposition to COPD, emphysema, or COPD and emphysema, and optionally a reference non-genetic database of non-genetic factors for predisposition to COPD, emphysema, or COPD and emphysema; and

a computer program embedded within the computer processor which, once data consisting of or including the result of a genetic analysis for which data is included in the reference genetic database is received, processes said data in the context of said reference databases to determine, as an outcome, the genetic state of the mammalian subject, said outcome being communicable once known, preferably to a user having input said data.

Preferably, said system is accessible via the internet or by personal computer.

In yet a further aspect, the invention provides a computer program suitable for use in a system as defined above comprising a computer usable medium having program code embodied in the medium for causing the computer program to process received data consisting of or including the result of at least one analysis of one or more genetic loci associated with predisposition to COPD, emphysema, or COPD and emphysema, in the context of both a reference genetic database of the results of said at least one genetic analysis and optionally a reference non-genetic database of non-genetic factors associated with predisposition to COPD, emphysema, or COPD and emphysema.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### DESCRIPTION OF THE PREFERRED EMBODIMENTS

Using case-control studies the frequencies of several genetic variants (polymorphisms) of candidate genes in smokers who have developed COPD, smokers who appear resistant to COPD, and blood donor controls have been compared. The majority of these candidate genes have confirmed (or likely) functional effects on gene expression or protein function. Specifically the frequencies of polymorphisms between blood donor controls, resistant smokers and those with COPD (subdivided into those with early onset and those with normal onset) have been compared. The present invention demonstrates that there are both protective and susceptibility polymorphisms present in selected candidate genes of the patients tested.

Specifically, 7 susceptibility genetic polymorphisms and 2 protective genetic polymorphisms have been identified. These are as follows:

| SNP ID | rs # | phenotype | genotype | OR | P value |
|---|---|---|---|---|---|
| Cer 1 | 10115703 | susceptible | GA/AA | 1.4 | 0.05 |
| XPD | 13181 | protective | GG | 0.65 | 0.01 |
| NAT2 | 1799930 | susceptible | GG | 1.3 | 0.05 |
| CYP2E1 | 2031920 | susceptible | CT/TT | 1.7 | 0.10 |
| IL-8 | 4073 | susceptible | TT | 1.5 | 0.002 |
| α1 | 4934 | susceptible | GG | 1.3 | 0.05 |
| anti-chymotrypsin | | | | | |
| FasL | 763110 | protective | TT | 0.8 | 0.11 |
| α5 nAChR | 16969968 | susceptible | AA | 1.5 | 0.06 |
| α5 nAChR | 1051730 | susceptible | TT | 1.6 | 0.02 |

A susceptibility genetic polymorphism is one which, when present, is indicative of an increased risk of developing COPD, emphysema, or both COPD and emphysema. In contrast, a protective genetic polymorphism is one which, when present, is indicative of a reduced risk of developing COPD, emphysema, or both COPD and emphysema.

As used herein, the phrase “risk of developing COPD, emphysema, or both COPD and emphysema” means the likelihood that a subject to whom the risk applies will develop COPD, emphysema, or both COPD and emphysema, and includes predisposition to, and potential onset of the disease. Accordingly, the phrase “increased risk of developing COPD, emphysema, or both COPD and emphysema” means that a subject having such an increased risk possesses an hereditary inclination or tendency to develop COPD, emphysema, or both COPD and emphysema. This does not mean that such a person will actually develop COPD, emphysema, or both COPD and emphysema at any time, merely that he or she has a greater likelihood of developing COPD, emphysema, or both COPD and emphysema compared to the general population of individuals that either does not possess a polymorphism associated with increased COPD, emphysema, or both COPD and emphysema risk, or does possess a polymorphism associated with decreased COPD, emphysema, or both COPD and emphysema risk. Subjects with an increased risk of developing COPD, emphysema, or both COPD and emphysema include those with a predisposition to COPD, emphysema, or both COPD and emphysema, such as a tendency or prediliction regardless of their lung function at the time of assessment, for example, a subject who is genetically inclined to COPD, emphysema, or both COPD and emphysema but who has normal lung function, those at potential risk, including subjects with a tendency to mildly reduced lung function who are likely to go on to suffer COPD, emphysema, or both COPD and emphysema if they keep smoking, and subjects with potential onset of COPD, emphysema, or both COPD and emphysema, who have a tendency to poor lung function on spirometry etc., consistent with COPD at the time of assessment.

Similarly, the phrase “decreased risk of developing COPD, emphysema, or both COPD and emphysema” means that a subject having such a decreased risk possesses an hereditary disinclination or reduced tendency to develop COPD, emphysema, or both COPD and emphysema. This does not mean that such a person will not develop COPD, emphysema, or both COPD and emphysema at any time, merely that he or she has a decreased likelihood of developing COPD, emphysema, or both COPD and emphysema compared to the general population of individuals that either does possess one or more polymorphisms associated with increased COPD, emphysema, or both COPD and emphysema risk, or does not possess a polymorphism associated with decreased COPD, emphysema, or both COPD and emphysema risk.

It will be understood that in the context of the present invention the term “polymorphism”means the occurrence together in the same population at a rate greater than that attributable to random mutation (usually greater than 1%) of two or more alternate forms (such as alleles or genetic markers) of a chromosomal locus that differ in nucleotide sequence or have variable numbers of repeated nucleotide units. See www.ornl.gov/sci/techresources/Human_Genome/publicat/97pr/09gloss.html#p.

Accordingly, the term “polymorphisms” is used herein contemplates genetic variations, including single nucleotide substitutions, insertions and deletions of nucleotides, repetitive sequences (such as microsatellites), and the total or partial absence of genes (eg. null mutations). As used herein, the term “polymorphisms” also includes genotypes and haplotypes. A genotype is the genetic composition at a specific locus or set of loci. A haplotype is a set of closely linked genetic markers present on one chromosome which are not easily separable by recombination, tend to be inherited together, and may be in linkage disequilibrium. A haplotype can be identified by patterns of polymorphisms such as SNPs. Similarly, the term “single nucleotide polymorphism” or “SNP” in the context of the present invention includes single base nucleotide subsitutions and short deletion and insertion polymorphisms.

A reduced or increased risk of a subject developing COPD, emphysema, or both COPD and emphysema may be diagnosed by analysing a sample from said subject for the presence or absence of a polymorphism selected from the group comprising, consisting essentially of, or consisting of:
rs10115703 G/A polymorphism in the gene encoding Cer 1;
rs13181 G/T polymorphism in the gene encoding XPD;
rs1799930 G/A polymorphism in the gene encoding NAT2;
rs2031920 C/T polymorphism in the gene encoding CYP2E1;
rs4073 T/A polymorphism in the gene encoding IL-8;
rs763110 C/T polymorphism in the gene encoding FasL;
rs16969968 G/A polymorphism in the gene encoding α5-nAChR;
rs1051730 C/T polymorphism in the gene encoding α5-nAChR;
or one or more polymorphisms which are in linkage disequilibrium with any one or more of the above group.

These polymorphisms can also be analysed in combinations of two or more, or in combination with other polymorphisms indicative of a subject's risk of developing COPD, emphysema, or both COPD and emphysema, inclusive of the remaining polymorphisms listed above.

Expressly contemplated are combinations of the above polymorphisms with polymorphisms as described in PCT International application PCT/NZ02/00106, published as WO 02/099134.

Also expressly contemplated are combinations of the above polymorphisms with polymorphisms as described in New Zealand Patent Applications No. 539934, No. 541935, No. 545283, and PCT International Application PCT/NZ2006/000103 (published as WO2006/121351) each incorporated herein in its entirety.

Assays which involve combinations of polymorphisms, including those amenable to high throughput, such as those utilising microarrays or mass spectometry, are preferred.

Statistical analyses, particularly of the combined effects of these polymorphisms, show that the genetic analyses of the present invention can be used to determine the risk quotient of any smoker and in particular to identify smokers at greater risk of developing COPD. Such combined analysis can be of combinations of susceptibility polymorphisms only, of protective polymorphisms only, or of combinations of both. Analysis can also be step-wise, with analysis of the presence or absence of protective polymorphisms occurring first and then with analysis of susceptibility polymorphisms proceeding only where no protective polymorphisms are present.

Thus, through systematic analysis of the frequency of these polymorphisms in well defined groups of smokers and non-smokers, as described herein, it is possible to implicate certain proteins in the development of COPD and improve the ability to identify which smokers are at increased risk of developing COPD-related impaired lung function and COPD for predictive purposes.

The present results show for the first time that the minority of smokers who develop COPD, emphysema, or both COPD and emphysema do so because they have one or more of the susceptibility polymorphisms and few or none of the protective polymorphisms defined herein. It is thought that the presence of one or more suscetptible polymorphisms, together with the damaging irritant and oxidant effects of smoking, combine to make this group of smokers highly susceptible to developing COPD, emphysema, or both COPD and emphysema. Additional risk factors, such as familial history, age, weight, pack years, etc., will also have an impact on the risk profile of a subject, and can be assessed in combination with the genetic analyses described herein.

It will be apparent to those skilled in the field that the convention of identifying promoter polymorphisms by their position relative to the +1 translation start site of the gene in which they occur is followed herein. Accordingly, the −765 C/G polymorphism in the promoter of the gene encoding Cyclooxygenase 2 described herein lies 765 nucleotides upstream of the +1 translation start site of the COX2 gene. The other polymorphisms disclosed herein are similarly identified with reference to the +1 translation start site.

The polymorphisms described herein can be detected directly or by detection of one or more polymorphisms which are in linkage disequilibrium with these polymorphisms. Linkage disequilibrium is a phenomenon in genetics whereby two or more mutations or polymorphisms are in such close genetic proximity that they are co-inherited. This means that in genotyping, detection of one polymorphism as present implies the presence of the other. (Reich D E et al; Linkage disequilibrium in the human genome, Nature 2001, 411:199-204.)

Various degrees of linkage disequilibrium are possible. Preferably, the one or more polymorphisms in linkage disequilibrium with one or more of the polymorphisms specified herein are in greater than about 60% linkage disequilibrium, are in about 70% linkage disequilibrium, about 75%, about 80%, about 85%, about 90%, about 91%, 92%, 93%, 94%, 95%, 96%, 97%, 98%, 99%, or about 100% linkage disequilibrium with one or more of the polymorphisms specified herein. Those skilled in the art will appreciate that linkage disequilibrium may also, when expressed with reference to the deviation of the observed frequency of a pair of alleles from the expected, be denoted by a capital D. Accordingly, the phrase “two alleles are in LD” usually means that D does not equal 0. Contrariwise, “linkage equilibrium” denotes the case D=0. When utilising this nomenclature, the one or more polymorphisms in LD with the one or more polymorphisms specified herein are preferably in LD of greater than about D′=0.6, of about D′=0.7, of about D′=0.75, of about D′=0.8, of about D′=0.85, of about D′=0.9, of about D′=0.91, of about D′=0.92, of about D′=0.93, of about D′=0.94, of about D′=0.95, of about D′=0.96, of about D′=0.97, of about D′=0.98, of about D′=0.99, or about D′=1.0. (Devlin and Risch 1995; A comparison of linkage disequilibrium measures for fine-scale mapping, Genomics 29: 311-322).

It will be apparent that polymorphsisms in linkage disequilibrium with one or more other polymorphism associated with increased or decreased risk of developing COPD, emphysema, or both COPD and emphysema will also provide utility as biomarkers for risk of developing COPD, emphysema, or both COPD and emphysema. The data presented herein shows that the frequency for SNPs in linkage disequilibrium is very similar, particularly when the degree of linkage disequilibrium is high, for example, at least about 80%, at least about 85%, at least about 90%, at least about 91%, 92%, 93%, 94%, 95%, 96%, 97%, 98%, 99%, or about 100% linkage disequilibrium. See, for example, the rs16969968 and rs1051730 polymorphisms in the nAChR gene, as shown in Table 14.

Accordingly, these genetically linked SNPs can be utilized in combined polymorphism analyses to derive a level of risk comparable to that calculated from the original SNP.

It will also be apparent that one or more polymorphisms in linkage disequilibrium with the polymorphisms specified herein can be identified, for example, using public data bases. Examples of such polymorphisms reported to be in linkage disequilibrium with the polymorphisms specified herein are presented in Table 15, and these and other examples may be found, for example, in the Genbank public database, or in HapMap.

There are numerous standard methods known in the art for determining whether a particular DNA sequence is present in a sample, many of which include the step of sequencing a DNA sample. Thus in one embodiment of the invention, the step determining whether or not the specified nucleotides are present in a nucleic acid derived from a subject, includes the step of sequencing the nucleic acid. Methods for nucleotide sequencing are well known to those skilled in the art.

An example of another art standard method known for determining whether a particular DNA sequence is present in a sample is the Polymerase Chain Reaction (PCR). A preferred aspect of the invention thus includes a step in which ascertaining whether a sequence comprising a polymorpism is present includes amplifying the DNA in the presence of sequence-specific primers, including allele-specific primers.

A primer of the present invention, used in PCR for example, is a nucleic acid molecule sufficiently complementary to the sequence on which it is based and of sufficient length to selectively hybridise to the corresponding portion of a nucleic acid molecule intended to be amplified and to prime synthesis thereof under in vitro conditions commonly used in PCR. Likewise, a probe of the present invention, is a molecule, for example a nucleic acid molecule of sufficient length and sufficiently complementary to the nucleic acid molecule of interest, which selectively binds under high or low stringency conditions with the nucleic acid sequence of interest for detection in the presence of nucleic acid molecules having differing sequences.

Accordingly, a preferred embodiment of the invention thus includes the step of amplifying a polynucleotide comprising a polymorphism in the presence of at least one primer comprising a nucleotide sequence of or complementary to the polymorphism or flanking sequence thereof, and/or in the presence of one or more primers comprising sequence flanking one of the polymorphisms selected from the group consisting of the rs10115703 G/A polymorphism in the gene encoding Cer 1, the rs 13181 G/T polymorphism in the gene encoding XPD, the rs1799930 G/A polymorphism in the gene encoding NAT2, the rs2031920 C/T polymorphism in the gene encoding CYP2E1, the rs4073 T/A polymorphism in the gene encoding IL-8, the rs763110 C/T polymorphism in the gene encoding FasL, the rs16969968 G/A polymorphism in the gene encoding α5-nAChR, the rs1051730 C/T polymorphism in the gene encoding α5-nAChR, or the rs4934 G/A polymorphism in the gene encoding α1 anti-chymotrypsin, and/or in the presence of one or more primers comprising sequence including one or other of the allele-specific polymorphic nucleotides at one of the polymorphism described above. PCR methods are well known by those skilled in the art (Mullis et al., 1994.) The template for amplification may be selected from genomic DNA, mRNA or first strand cDNA derived from a sample obtained from the mammalian subject under test (Sambrook et al., 1987).

Primers suitable for use in PCR based methods of the invention should be sufficiently complementary to the gene sequence or flanking sequence thereof, and of sufficient length to selectively hybridise to the corresponding portion of a nucleic acid molecule intended to be amplified and to prime synthesis thereof under in vitro conditions commonly used in PCR. Such primers should comprise at least about 12 contiguous bases. Examples of such PCR primers are presented herein.

Suitable PCR primers for use on a mammalian subject may include sequence corresponding to the allele-specific nucleotides described herein. Generation of a corresponding PCR product, or the lack of product, may constitute a test for the presence or absence of the specified nucleotides in the gene of the test subject.

Other methods for determining whether a particular nucleotide sequence is present in a sample may include the step of restriction enzyme digestion of nucleotide sample. Separation and visualisation of the digested restriction fragments by methods well known in the art, may form a diagnostic test for the presence of a particular nucleotide sequence. The nucleotide sequence digested may be a PCR product amplified as described above.

Still other methods for determining whether a particular nucleotide sequence is present in a sample include a step of hybridisation of a probe to a sample nucleotide sequence. Thus, methods for detecting for example the G allele-specific nucleotide at the rs10115703 G/A polymorphism in the gene encoding Cer 1 may comprise the additional steps of hybridisation of a probe derived from the Cer 1 gene.

Such probes should comprise a nucleic acid molecule of sufficient length and sufficiently complementary to the gene sequence, to selectively bind under high or low stringency conditions with the nucleic acid sequence of a sample to facilitate detection of the presence or absence of the allele-specific nucleotides described herein.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., 1987; Ausubel et al., 1987). Tm for polynucleotide molecules greater than about 100 bases can be calculated by the formula Tm=81.5+0.41% (G+C−log (Na+).

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length) ° C.

Such a probe may be hybridised with genomic DNA, mRNA, or cDNA produced from mRNA, derived from a sample taken from a mammalian subject under test. Such probes would typically comprise at least 12 contiguous nucleotides of or complementary to the gene sequence.

Such probes may additionally comprise means for detecting the presence of the probe when bound to sample nucleotide sequence. Methods for labelling probes such as radiolabelling are well known in the art (see for example, Sambrook et al., 1987).

The methods of the invention are primarily directed to the detection and identification of the above polymorphisms associated with COPD, which are all single nucleotide polymorphisms. In general terms, a single nucleotide polymorphism (SNP) is a single base change or point mutation resulting in genetic variation between individuals. SNPs occur in the human genome approximately once every 100 to 300 bases, and can occur in coding or non-coding regions. Due to the redundancy of the genetic code, a SNP in the coding region may or may not change the amino acid sequence of a protein product. A SNP in a non-coding region can, for example, alter gene expression by, for example, modifying control regions such as promoters, transcription factor binding sites, processing sites, ribosomal binding sites, and affect gene transcription, processing, and translation.

SNPs can facilitate large-scale association genetics studies, and there has recently been great interest in SNP discovery and detection. SNPs show great promise as markers for a number of phenotypic traits (including latent traits), such as for example, disease propensity and severity, wellness propensity, and drug responsiveness including, for example, susceptibility to adverse drug reactions. Knowledge of the association of a particular SNP with a phenotypic trait, coupled with the knowledge of whether an individual has said particular SNP, can enable the targeting of diagnostic, preventative and therapeutic applications to allow better disease management, to enhance understanding of disease states and to ultimately facilitate the discovery of more effective treatments, such as personalised treatment regimens.

Indeed, a number of databases have been constructed of known SNPs, and for some such SNPs, the biological effect associated with a SNP. For example, the NCBI SNP database “dbSNP” is incorporated into NCBI's Entrez system and can be queried using the same approach as the other Entrez databases such as PubMed and GenBank. This database has records for over 3.5 million reference SNPs mapped onto the human genome sequence. Each dbSNP entry includes the sequence context of the polymorphism (i.e., the surrounding sequence), the occurrence frequency of the polymorphism (by population or individual), and the experimental method(s), protocols, and conditions used to assay the variation, and can include information associating a SNP with a particular phenotypic trait.

At least in part because of the potential impact on health and wellness, there has been and continues to be a great deal of effort to develop methods that reliably and rapidly identify SNPs. This is no trivial task, at least in part because of the complexity of human genomic DNA, with a haploid genome of 3×10⁹ base pairs, and the associated sensitivity and discriminatory requirements.

Genotyping approaches to detect SNPs well-known in the art include DNA sequencing, methods that require allele specific hybridization of primers or probes, allele specific incorporation of nucleotides to primers bound close to or adjacent to the polymorphisms (often referred to as “single base extension”, or “minisequencing”), allele-specific ligation (joining) of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation is also possible where the SNP lies in a coding region and results in an amino acid change.

DNA sequencing allows the direct determination and identification of SNPs. The benefits in specificity and accuracy are generally outweighed for screening purposes by the difficulties inherent in whole genome, or even targeted subgenome, sequencing.

Mini-sequencing involves allowing a primer to hybridize to the DNA sequence adjacent to the SNP site on the test sample under investigation. The primer is extended by one nucleotide using all four differentially tagged fluorescent dideoxynucleotides (A,C,G, or T), and a DNA polymerase. Only one of the four nucleotides (homozygous case) or two of the four nucleotides (heterozygous case) is incorporated. The base that is incorporated is complementary to the nucleotide at the SNP position.

A number of sequencing methods and platforms are particularly suited to large-scale implementation, and are amenable to use in the methods of the invention. These include pyrosequencing methods, such as that utilised in the GS FLX pyrosequencing platform available from 454 Life Sciences (Branford, Conn.) which can generate 100 million nucleotide data in a 7.5 hour run with a single machine, and solid-state sequencing methods, such as that utilised in the SOLiD sequencing platform (Applied Biosystems, Foster City, Calif.).

A number of methods currently used for SNP detection involve site-specific and/or allele-specific hybridisation. These methods are largely reliant on the discriminatory binding of oligonucleotides to target sequences containing the SNP of interest. The techniques of Illumina (San Diego, Calif.), Affymetrix (Santa Clara, Calif.) and Nanogen Inc. (San Diego, Calif.) are particularly well-known, and utilize the fact that DNA duplexes containing single base mismatches are much less stable than duplexes that are perfectly base-paired. The presence of a matched duplex is usually detected by fluorescence. A number of whole-genome genotyping products and solutions amenable or adaptable for use in the present invention are now available, including those available from the above companies.The majority of methods to detect or identify SNPs by site-specific hybridisation require target amplification by methods such as PCR to increase sensitivity and specificity (see, for example U.S. Pat. No. 5,679,524, PCT publication WO 98/59066, PCT publication WO 95/12607). US Patent Application publication number 20050059030 (incorporated herein in its entirety) describes a method for detecting a single nucleotide polymorphism in total human DNA without prior amplification or complexity reduction to selectively enrich for the target sequence, and without the aid of any enzymatic reaction. The method utilises a single-step hybridization involving two hybridization events: hybridization of a first portion of the target sequence to a capture probe, and hybridization of a second portion of said target sequence to a detection probe. Both hybridization events happen in the same reaction, and the order in which hybridisation occurs is not critical.

US Patent Application publication number 20050042608 (incorporated herein in its entirety) describes a modification of the method of electrochemical detection of nucleic acid hybridization of Thorp et al. (U.S. Pat. No. 5,871,918). Briefly, capture probes are designed, each of which has a different SNP base and a sequence of probe bases on each side of the SNP base. The probe bases are complementary to the corresponding target sequence adjacent to the SNP site. Each capture probe is immobilized on a different electrode having a non-conductive outer layer on a conductive working surface of a substrate. The extent of hybridization between each capture probe and the nucleic acid target is detected by detecting the oxidation-reduction reaction at each electrode, utilizing a transition metal complex. These differences in the oxidation rates at the different electrodes are used to determine whether the selected nucleic acid target has a single nucleotide polymorphism at the selected SNP site.

The technique of Lynx Therapeutics (Hayward, Calif.) using MEGATYPE™ technology can genotype very large numbers of SNPs simultaneously from small or large pools of genomic material. This technology uses fluorescently labeled probes and compares the collected genomes of two populations, enabling detection and recovery of DNA fragments spanning SNPs that distinguish the two populations, without requiring prior SNP mapping or knowledge.

A number of other methods for detecting and identifying SNPs exist. These include the use of mass spectrometry, for example, to measure probes that hybridize to the SNP. This technique varies in how rapidly it can be performed, from a few samples per day to a high throughput of many thousands of SNPs per day, using mass code tags. A preferred example is the use of mass spectrometric determination of a nucleic acid sequence which comprises the polymorphisms of the invention, for example, which includes the Cerberus 1 gene or a complementary sequence. Such mass spectrometric methods are known to those skilled in the art, and the genotyping methods of the invention are amenable to adaptation for the mass spectrometric detection of the polymorphisms of the invention, for example, the Cerberus 1 polymorphism of the invention.

SNPs can also be determined by ligation-bit analysis. This analysis requires two primers that hybridize to a target with a one nucleotide gap between the primers. Each of the four nucleotides is added to a separate reaction mixture containing DNA polymerase, ligase, target DNA and the primers. The polymerase adds a nucleotide to the 3′end of the first primer that is complementary to the SNP, and the ligase then ligates the two adjacent primers together. Upon heating of the sample, if ligation has occurred, the now larger primer will remain hybridized and a signal, for example, fluorescence, can be detected. A further discussion of these methods can be found in U.S. Pat. Nos. 5,919,626; 5,945,283; 5,242,794; and 5,952,174.

U.S. Pat. No. 6,821,733 (incorporated herein in its entirety) describes methods to detect differences in the sequence of two nucleic acid molecules that includes the steps of: contacting two nucleic acids under conditions that allow the formation of a four-way complex and branch migration; contacting the four-way complex with a tracer molecule and a detection molecule under conditions in which the detection molecule is capable of binding the tracer molecule or the four-way complex; and determining binding of the tracer molecule to the detection molecule before and after exposure to the four-way complex. Competition of the four-way complex with the tracer molecule for binding to the detection molecule indicates a difference between the two nucleic acids.

Protein- and proteomics-based approaches are also suitable for polymorphism detection and analysis. Polymorphisms which result in or are associated with variation in expressed proteins can be detected directly by analysing said proteins. This typically requires separation of the various proteins within a sample, by, for example, gel electrophoresis or HPLC, and identification of said proteins or peptides derived therefrom, for example by NMR or protein sequencing such as chemical sequencing or more prevalently mass spectrometry. Proteomic methodologies are well known in the art, and have great potential for automation. For example, integrated systems, such as the ProteomIQ™ system from Proteome Systems, provide high throughput platforms for proteome analysis combining sample preparation, protein separation, image acquisition and analysis, protein processing, mass spectrometry and bioinformatics technologies.

The majority of proteomic methods of protein identification utilise mass spectrometry, including ion trap mass spectrometry, liquid chromatography (LC) and LC/MSn mass spectrometry, gas chromatography (GC) mass spectroscopy, Fourier transform-ion cyclotron resonance-mass spectrometer (FT-MS), MALDI-TOF mass spectrometry, and ESI mass spectrometry, and their derivatives. Mass spectrometric methods are also useful in the determination of post-translational modification of proteins, such as phosphorylation or glycosylation, and thus have utility in determining polymorphisms that result in or are associated with variation in post-translational modifications of proteins.

Associated technologies are also well known, and include, for example, protein processing devices such as the “Chemical Inkjet Printer” comprising piezoelectric printing technology that allows in situ enzymatic or chemical digestion of protein samples electroblotted from 2-D PAGE gels to membranes by jetting the enzyme or chemical directly onto the selected protein spots. After in-situ digestion and incubation of the proteins, the membrane can be placed directly into the mass spectrometer for peptide analysis.

A large number of methods reliant on the conformational variability of nucleic acids have been developed to detect SNPs.

For example, Single Strand Conformational Polymorphism (SSCP, Orita et al., PNAS 1989 86:2766-2770) is a method reliant on the ability of single-stranded nucleic acids to form secondary structure in solution under certain conditions. The secondary structure depends on the base composition and can be altered by a single nucleotide substitution, causing differences in electrophoretic mobility under nondenaturing conditions. The various polymorphs are typically detected by autoradiography when radioactively labelled, by silver staining of bands, by hybridisation with detectably labelled probe fragments or the use of fluorescent PCR primers which are subsequently detected, for example by an automated DNA sequencer.

Modifications of SSCP are well known in the art, and include the use of differing gel running conditions, such as for example differing temperature, or the addition of additives, and different gel matrices. Other variations on SSCP are well known to the skilled artisan, including, RNA-SSCP, restriction endonuclease fingerprinting-SSCP, dideoxy fingerprinting (a hybrid between dideoxy sequencing and SSCP), bi-directional dideoxy fingerprinting (in which the dideoxy termination reaction is performed simultaneously with two opposing primers), and Fluorescent PCR-SSCP (in which PCR products are internally labelled with multiple fluorescent dyes, may be digested with restriction enzymes, followed by SSCP, and analysed on an automated DNA sequencer able to detect the fluorescent dyes).

Other methods which utilise the varying mobility of different nucleic acid structures include Denaturing Gradient Gel Electrophoresis (DGGE), Temperature Gradient Gel Electrophoresis (TGGE), and Heteroduplex Analysis (HET). Here, variation in the dissociation of double stranded DNA (for example, due to base-pair mismatches) results in a change in electrophoretic mobility. These mobility shifts are used to detect nucleotide variations.

Denaturing High Pressure Liquid Chromatography (HPLC) is yet a further method utilised to detect SNPs, using HPLC methods well-known in the art as an alternative to the separation methods described above (such as gel electophoresis) to detect, for example, homoduplexes and heteroduplexes which elute from the HPLC column at different rates, thereby enabling detection of mismatch nucleotides and thus SNPs.

Yet further methods to detect SNPs rely on the differing susceptibility of single stranded and double stranded nucleic acids to cleavage by various agents, including chemical cleavage agents and nucleolytic enzymes. For example, cleavage of mismatches within RNA:DNA heteroduplexes by RNase A, of heteroduplexes by, for example bacteriophage T4 endonuclease YII or T7 endonuclease I, of the 5′ end of the hairpin loops at the junction between single stranded and double stranded DNA by cleavase I, and the modification of mispaired nucleotides within heteroduplexes by chemical agents commonly used in Maxam-Gilbert sequencing chemistry, are all well known in the art.

Further examples include the Protein Translation Test (PTT), used to resolve stop codons generated by variations which lead to a premature termination of translation and to protein products of reduced size, and the use of mismatch binding proteins. Variations are detected by binding of, for example, the MutS protein, a component of Escherichia coli DNA mismatch repair system, or the human hMSH2 and GTBP proteins, to double stranded DNA heteroduplexes containing mismatched bases. DNA duplexes are then incubated with the mismatch binding protein, and variations are detected by mobility shift assay. For example, a simple assay is based on the fact that the binding of the mismatch binding protein to the heteroduplex protects the heteroduplex from exonuclease degradation.

Those skilled in the art will know that a particular SNP, particularly when it occurs in a regulatory region of a gene such as a promoter, can be associated with altered expression of a gene. Altered expression of a gene can also result when the SNP is located in the coding region of a protein-encoding gene, for example where the SNP is associated with codons of varying usage and thus with tRNAs of differing abundance. Such altered expression can be determined by methods well known in the art, and can thereby be employed to detect such SNPs. Similarly, where a SNP occurs in the coding region of a gene and results in a non-synonomous amino acid substitution, such substitution can result in a change in the function of the gene product. Similarly, in cases where the gene product is an RNA, such SNPs can result in a change of function in the RNA gene product. Any such change in function, for example as assessed in an activity or functionality assay, can be employed to detect such SNPs.

The above methods of detecting and identifying SNPs are amenable to use in the methods of the invention.

Of course, in order to detect and identify SNPs in accordance with the invention, a sample containing material to be tested is obtained from the subject. The sample can be any sample potentially containing the target SNPs (or target polypeptides, as the case may be) and obtained from any bodily fluid (blood, urine, saliva, etc) biopsies or other tissue preparations.

DNA or RNA can be isolated from the sample according to any of a number of methods well known in the art. For example, methods of purification of nucleic acids are described in Tijssen; Laboratory Techniques in Biochemistry and Molecular Biology: Hybridization with nucleic acid probes Part 1: Theory and Nucleic acid preparation, Elsevier, New York, N.Y. 1993, as well as in Maniatis, T., Fritsch, E. F. and Sambrook, J., Molecular Cloning Manual 1989.

To assist with detecting the presence or absence of polymorphisms/SNPs, nucleic acid probes and/or primers can be provided. Such probes have nucleic acid sequences specific for chromosomal changes evidencing the presence or absence of the polymorphism and are preferably labeled with a substance that emits a detectable signal when combined with the target polymorphism.

The nucleic acid probes can be genomic DNA or cDNA or mRNA, or any RNA-like or DNA-like material, such as peptide nucleic acids, branched DNAs, and the like. The probes can be sense or antisense polynucleotide probes. Where target polynucleotides are double-stranded, the probes may be either sense or antisense strands. Where the target polynucleotides are single-stranded, the probes are complementary single strands.

The probes can be prepared by a variety of synthetic or enzymatic schemes, which are well known in the art. The probes can be synthesized, in whole or in part, using chemical methods well known in the art (Caruthers et al., Nucleic Acids Res., Symp. Ser., 215-233 (1980)). Alternatively, the probes can be generated, in whole or in part, enzymatically.

Nucleotide analogs can be incorporated into probes by methods well known in the art. The only requirement is that the incorporated nucleotide analog must serve to base pair with target polynucleotide sequences. For example, certain guanine nucleotides can be substituted with hypoxanthine, which base pairs with cytosine residues. However, these base pairs are less stable than those between guanine and cytosine. Alternatively, adenine nucleotides can be substituted with 2,6-diaminopurine, which can form stronger base pairs than those between adenine and thymidine.

Additionally, the probes can include nucleotides that have been derivatized chemically or enzymatically. Typical chemical modifications include derivatization with acyl, alkyl, aryl or amino groups.

The probes can be immobilized on a substrate. Preferred substrates are any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which the polynucleotide probes are bound. Preferably, the substrates are optically transparent.

Furthermore, the probes do not have to be directly bound to the substrate, but rather can be bound to the substrate through a linker group. The linker groups are typically about 6 to 50 atoms long to provide exposure to the attached probe. Preferred linker groups include ethylene glycol oligomers, diamines, diacids and the like. Reactive groups on the substrate surface react with one of the terminal portions of the linker to bind the linker to the substrate. The other terminal portion of the linker is then functionalized for binding the probe.

The probes can be attached to a substrate by dispensing reagents for probe synthesis on the substrate surface or by dispensing preformed DNA fragments or clones on the substrate surface. Typical dispensers include a micropipette delivering solution to the substrate with a robotic system to control the position of the micropipette with respect to the substrate. There can be a multiplicity of dispensers so that reagents can be delivered to the reaction regions simultaneously.

Nucleic acid microarrays are preferred. Such microarrays (including nucleic acid chips) are well known in the art (see, for example U.S. Pat. Nos. 5,578,832; 5,861,242; 6,183,698; 6,287,850; 6,291,183; 6,297,018; 6,306,643; and 6,308,170, each incorporated by reference).

Alternatively, antibody microarrays can be produced. The production of such microarrays is essentially as described in Schweitzer & Kingsmore, “Measuring proteins on microarrays”, Curr Opin Biotechnol 2002; 13(1): 14-9; Avseekno et al, “Immobilization of proteins in immunochemical microarrays fabricated by electrospray deposition”, Anal Chem 2001 15; 73(24): 6047-52; Huang, “Detection of multiple proteins in an antibody-based protein microarray system, Immunol Methods 2001 1; 255 (1-2): 1-13.

The present invention also contemplates the preparation of kits for use in accordance with the present invention. Suitable kits include various reagents for use in accordance with the present invention in suitable containers and packaging materials, including tubes, vials, and shrink-wrapped and blow-molded packages.

Materials suitable for inclusion in an exemplary kit in accordance with the present invention comprise one or more of the following: gene specific PCR primer pairs (oligonucleotides) that anneal to DNA or cDNA sequence domains that flank the genetic polymorphisms of interest, reagents capable of amplifying a specific sequence domain in either genomic DNA or cDNA without the requirement of performing PCR; reagents required to discriminate between the various possible alleles in the sequence domains amplified by PCR or non-PCR amplification (e.g., restriction endonucleases, oligonucleotide that anneal preferentially to one allele of the polymorphism, including those modified to contain enzymes or fluorescent chemical groups that amplify the signal from the oligonucleotide and make discrimination of alleles more robust); reagents required to physically separate products derived from the various alleles (e.g. agarose or polyacrylamide and a buffer to be used in electrophoresis, HPLC columns, SSCP gels, formamide gels or a matrix support for MALDI-TOF).

It will be appreciated that the methods of the invention can be performed in conjunction with an analysis of other risk factors known to be associated with COPD, emphysema, or both COPD and emphysema. Such risk factors include epidemiological risk factors associated with an increased risk of developing COPD, emphysema, or both COPD and emphysema. Such risk factors include, but are not limited to smoking and/or exposure to tobacco smoke, age, sex and familial history. These risk factors can be used to augment an analysis of one or more polymorphisms as herein described when assessing a subject's risk of developing chronic obstructive pulmonary disease (COPD) and/or emphysema.

The invention further provides diagnostic kits useful in determining the allelic profile of mammalian subjects, for example for use in the methods of the present invention.

Accordingly, in one embodiment the invention provides a diagnostic kit which can be used to determine the genotype of a mammalian subject's genetic material at one or more of the polymorphism of the invention. One kit includes a set of primers used for amplifying the genetic material. A kit can contain a primer including a nucleotide sequence for amplifying a region of the genetic material containing one of the naturally occurring mutations described herein. Such a kit could also include a primer for amplifying the corresponding region of the normal gene that produces a functionally wild type protein. Usually, such a kit would also include another primer upstream or downstream of the region of the gene comprising the polymorphism. These primers are used to amplify the segment containing the mutation of interest. The actual genotyping is carried out using primers that target specific mutations described herein and that could function as allele-specific oligonucleotides in conventional hybridisation, Taqman assays, OLE assays, etc. Alternatively, primers can be designed to permit genotyping by microsequencing.

One kit of primers can include first, second and third primers, (a), (b) and (c), respectively. Primer (a) is based on a region containing a mutation such as described above. Primer (b) encodes a region upstream or downstream of the region to be amplified by a primer (a) so that genetic material containing the mutation is amplified, by PCR, for example, in the presence of the two primers. Primer (c) is based on the region corresponding to that on which primer (a) is based, but lacking the mutation. Thus, genetic material containing the non-mutated region will be amplified in the presence of primers (b) and (c). Genetic material homozygous for the wild type gene will thus provide amplified products in the presence of primers (b) and (c). Genetic material homozygous for the mutated gene will thus provide amplified products in the presence of primers (a) and (b). Heterozygous genetic material will provide amplified products in both cases.

For example, the kit may include a primer comprising a guanine at the position corresponding to the rs16969968 G/A polymorphism in the nAChR gene or comprising a nucleotide capable of hybridising to a guanine at the position corresponding to the rs16969968 G/A polymorphism in the nAChR gene. Those skilled in the art will recognise that in such a primer, the guanine, or the nucleotide capable of hybridising to a guanine, as applicable, may be substituted for a nucleotide analogue having the same discriminatory base-pairing as the substituted nucleotide.

In another example, the kit may include a primer comprising a adenine at the position corresponding to the rs16969968 G/A polymorphism in the nAChR gene, or comprising a nucleotide capable of hybridising to a adenine at the position corresponding to the rs16969968 G/A polymorphism in the nAChR gene. Those skilled in the art will recognise that in such a primer, the thymine, or the nucleotide capable of hybridising to a thymine, as applicable, may be substituted for a nucleotide analogue having the same discriminatory base-pairing as the substituted nucleotide.

Those skilled in the art will appreciate that the invention provides kits comprising primers similarly directed to the other polymorphisms specified herein.

In one embodiment, the diagnostic kit is useful in detecting DNA comprising a variant gene or encoding a variant polypeptide at least partially lacking wild type activity in a mammalian subject which includes first and second primers for amplifying the DNA, the primers being complementary to nucleotide sequences of the DNA upstream and downstream, respectively, of a polymorphism in the gene which results in decreased or increased risk of COPD, emphysema, or both COPD and emphysema, preferably wherein at least one of the nucleotide sequences is selected to be from a non-coding region of the gene. The kit can also include a third primer complementary to a naturally occurring mutation of a coding portion of the wild type gene. Preferably the kit includes instructions for use, for example in accordance with a method of the invention.

In one embodiment, the diagnostic kit comprises a nucleotide probe complementary to the sequence comprising the polymorphism, or an oligonucleotide fragment thereof, for example, for hybridisation with mRNA from a sample of cells; and means for detecting the nucleotide probe bound to mRNA in the sample with a standard. In a particular aspect, the kit of this aspect of the invention includes a probe having a nucleic acid molecule sufficiently complementary with a sequence of a gene described herein or complements thereof, so as to bind thereto under stringent conditions. “Stringent hybridisation conditions” takes on its common meaning to a person skilled in the art. Appropriate stringency conditions which promote nucleic acid hybridisation, for example, 6× sodium chloride/sodium citrate (SSC) at about 45° C. are known to those skilled in the art, including in Current Protocols in Molecular Biology, John Wiley & Sons, NY (1989). Appropriate wash stringency depends on degree of homology and length of probe. If homology is 100%, a high temperature (65° C. to 75° C.) may be used. However, if the probe is very short (<100 bp), lower temperatures must be used even with 100% homology. In general, one starts washing at low temperatures (37° C. to 40° C.), and raises the temperature by 3-5° C. intervals until background is low enough to be a major factor in autoradiography. The diagnostic kit can also contain an instruction manual for use of the kit.

The invention also includes kits for detecting the presence of protein encoded by a gene as described herein in a biological sample. For example, the kit can include a compound or agent capable of detecting Cerberus 1 protein in a biological sample; and a standard. The compound or agent can be packaged in a suitable container. The kit can further comprise instructions for using the kit to detect the protein.

In one embodiment, the diagnostic kit comprises an antibody or an antibody composition useful for detection of the presence or absence of wild type protein and/or the presence or absence of a variant protein at least partially lacking wild type activity, together with instructions for use, for example in a method of the invention.

For antibody-based kits, the kit can include: (1) a first antibody (e.g., attached to a solid support) which binds to a polypeptide corresponding to a marker; and, optionally, (2) a second, different antibody which binds to either the polypeptide or the first antibody and is conjugated to a detectable agent.

The kit can also include a buffering agent, a preservative, or a protein stabilizing agent. The kit can also include components necessary for detecting the detectable agent (e.g., an enzyme or a substrate). The kit can also contain a control sample or a series of control samples which can be assayed and compared to the test sample contained. Each component of the kit can be enclosed within an individual container and all of the various containers can be within a single package, along with instructions for interpreting the results of the assays performed using the kit.

Sample Preparation

As will be apparent to persons skilled in the art, samples suitable for use in the methods of the present invention may be obtained from tissues or fluids as convenient, and so that the sample contains the moiety or moieties to be tested. For example, where nucleic acid is to be analysed, tissues or fluids containing nucleic acid will be used.

Conveniently, samples may be taken from milk, tissues, blood, serum, plasma, cerebrospinal fluid, urine, semen or saliva. Tissue samples may be obtained using standard techniques such as cell scrapings or biopsy techniques. For example, the cell or tissue samples may be obtained by using an ear punch to collect ear tissue from non-human mammalian subjects. Similarly, blood sampling is routinely performed, for example for pathogen testing, and methods for taking blood samples are well known in the art. Likewise, methods for storing and processing biological samples are well known in the art. For example, tissue samples may be frozen until tested if required. In addition, one of skill in the art would realize that some test samples would be more readily analyzed following a fractionation or purification procedure, for example, separation of whole blood into serum or plasma components.

Computer-Related Embodiments

It will also be appreciated that the methods of the invention are amenable to use with and the results analysed by computer systems, software and processes. Computer systems, software and processes to identify and analyse genetic polymorphisms are well known in the art. Similarly, implementation of the algorithm utilised to generate a SNP score as described herein in computer systems, software and processes is also contemplated. For example, the results of one or more genetic analyses as described herein may be analysed using a computer system and processed by such a system utilising a computer-executable example of the algorithm described herein.

Both the SNPs and the results of an analysis of the SNPs utilised in the present invention may be “provided” in a variety of mediums to facilitate use thereof. As used in this section, “provided” refers to a manufacture, other than an isolated nucleic acid molecule, that contains SNP information of the present invention. Such a manufacture provides the SNP information in a form that allows a skilled artisan to examine the manufacture using means not directly applicable to examining the SNPs or a subset thereof as they exist in nature or in purified form. The SNP information that may be provided in such a form includes any of the SNP information provided by the present invention such as, for example, polymorphic nucleic acid and/or amino acid sequence information, information about observed SNP alleles, alternative codons, populations, allele frequencies, SNP types, and/or affected proteins, identification as a protective SNP or a susceptibility SNP, weightings (for example for use in an algorithm utilised to derive a SNP score as described herein), or any other information provided by the present invention in Tables 1-15 and/or the Sequence ID Listing.

In one application of this embodiment, the SNPs and the results of an analysis of the SNPs utilised in the present invention can be recorded on a computer readable medium. As used herein, “computer readable medium” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising computer readable medium having recorded thereon SNP information of the present invention. One such medium is provided with the present application, namely, the present application contains computer readable medium (floppy disc) that has nucleic acid sequences used in analysing the SNPs utilised in the present invention provided/recorded thereon in ASCII text format in a Sequence Listing along with accompanying Tables that contain detailed SNP and sequence information.

As used herein, “recorded” refers to a process for storing information on computer readable medium. A skilled artisan can readily adopt any of the presently known methods for recording information on computer readable medium to generate manufactures comprising the SNP information of the present invention.

A variety of data storage structures are available to a skilled artisan for creating a computer readable medium having recorded thereon SNP information of the present invention. The choice of the data storage structure will generally be based on the means chosen to access the stored information. In addition, a variety of data processor programs and formats can be used to store the SNP information of the present invention on computer readable medium. For example, sequence information can be represented in a word processing text file, formatted in commercially-available software such as WordPerfect and Microsoft Word, represented in the form of an ASCII file, or stored in a database application, such as OB2, Sybase, Oracle, or the like. A skilled artisan can readily adapt any number of data processor structuring formats (e.g., text file or database) in order to obtain computer readable medium having recorded thereon the SNP information of the present invention.

By providing the SNPs and/or the results of an analysis of the SNPs utilised in the present invention in computer readable form, a skilled artisan can routinely access the SNP information for a variety of purposes. Computer software is publicly available which allows a skilled artisan to access sequence information provided in a computer readable medium. Examples of publicly available computer software include BLAST (Altschul et at, J. Mol. Biol. 215:403-410 (1990)) and BLAZE (Brutlag et at, Comp. Chem. 17:203-207 (1993)) search algorithms.

The present invention further provides systems, particularly computer-based systems, which contain the SNP information described herein. Such systems may be designed to store and/or analyze information on, for example, a number of SNP positions, or information on SNP genotypes from a number of individuals. The SNP information of the present invention represents a valuable information source. The SNP information of the present invention stored/analyzed in a computer-based system may be used for such applications as identifying subjects at risk of COPD, in addition to computer-intensive applications as determining or analyzing SNP allele frequencies in a population, mapping disease genes, genotype-phenotype association studies, grouping SNPs into haplotypes, correlating SNP haplotypes with response to particular drugs, or for various other bioinformatic, pharmacogenomic, drug development, or human identification/forensic applications.

As used herein, “a computer-based system” refers to the hardware, software, and data storage used to analyze the SNP information of the present invention. The minimum hardware of the computer-based systems of the present invention typically comprises a central processing unit (CPU), an input, an output, and data storage. A skilled artisan can readily appreciate that any one of the currently available computer-based systems are suitable for use in the present invention. Such a system can be changed into a system of the present invention by utilizing the SNP information, such as that provided herewith on the floppy disc, or a subset thereof, without any experimentation.

As stated above, the computer-based systems of the present invention comprise data storage having stored therein SNP information, such as SNPs and/or the results of an analysis of the SNPs utilised in the present invention, and the necessary hardware and software for supporting and implementing one or more programs or algorithms. As used herein, “data storage” refers to memory which can store SNP information of the present invention, or a memory access facility which can access manufactures having recorded thereon the SNP information of the present invention.

The one or more programs or algorithms are implemented on the computer-based system to identify or analyze the SNP information stored within the data storage. For example, such programs or algorithms can be used to determine which nucleotide is present at a particular SNP position in a target sequence, to analyse the results of a genetic analysis of the SNPs described herein, or to derive a SNP score as described herein. As used herein, a “target sequence” can be any DNA sequence containing the SNP position(s) to be analysed, searched or queried.

A variety of structural formats for the input and output can be used to input and output the information in the computer-based systems of the present invention. An exemplary format for an output is a display that depicts the SNP information, such as the presence or absence of specified nucleotides (alleles) at particular SNP positions of interest, or the derived SNP score for a subject. Such presentation can provide a rapid, binary scoring system for many SNPs or subjects simultaneously. It will be appreciated that such output may be accessed remotely, for example over a LAN or the internet. Typically, given the nature of SNP information, such remote accessing of such output or of the computer system itself is available only to verified users so that the security of the SNP information and/or the computer system is maintained. Methods to control access to computer systems and the data residing thereon are well-known in the art, and are amenable to the embodiments of the present invention.

One exemplary embodiment of a computer-based system comprising SNP information of the present invention that can be used to implement the present invention includes a processor connected to a bus. Also connected to the bus are a main memory (preferably implemented as random access memory, RAM) and a variety of secondary storage devices, such as a hard drive and a removable medium storage device. The removable medium storage device may represent, for example, a floppy disc drive, a CD-ROM drive, a magnetic tape drive, etc. A removable storage medium (such as a floppy disc, a compact disc, a magnetic tape, etc.) containing control logic and/or data recorded therein may be inserted into the removable medium storage device. The computer system includes appropriate software for reading the control logic and/or the data from the removable storage medium once inserted in the removable medium storage device. The SNP information of the present invention may be stored in a well-known manner in the main memory, any of the secondary storage devices, and/or a removable storage medium. Software for accessing and processing the SNP information (such as SNP scoring tools, search tools, comparing tools, etc.) preferably resides in main memory during execution.

Accordingly, the present invention provides a system for determining a subject's risk of developing COPD, emphysema, or both COPD and emphysema, said system comprising:

computer processor means for receiving, processing and communicating data;

storage means for storing data including a reference genetic database of the results of at least one genetic analysis with respect to COPD, emphysema, or both COPD and emphysema and optionally a reference non-genetic database of non-genetic risk factors for COPD, emphysema, or both COPD and emphysema; and

a computer program embedded within the computer processor which, once data consisting of or including the result of a genetic analysis for which data is included in the reference genetic database is received, processes said data in the context of said reference databases to determine, as an outcome, the subject's risk of developing COPD, emphysema, or both COPD and emphysema, said outcome being communicable once known, preferably to a user having input said data.

Preferably, the at least one genetic analysis is an analysis of one or more polymorphisms selected from the group comprising, consisting essentially of, or consisting of:
rs10115703 G/A polymorphism in the gene encoding Cer 1;
rs13181 G/T polymorphism in the gene encoding XPD;
rs1799930 G/A polymorphism in the gene encoding NAT2;
rs2031920 C/T polymorphism in the gene encoding CYP2E1;
rs4073 T/A polymorphism in the gene encoding IL-8;
rs763110 C/T polymorphism in the gene encoding FasL;
rs16969968 G/A polymorphism in the gene encoding α5-nAChR;
rs1051730 C/T polymorphism in the gene encoding α5-nAChR;
rs4934 G/A polymorphism in the gene encoding α1 anti-chymotrypsin;
the rs1489759 A/G polymorphism in the gene encoding HHIP;
the rs2202507 A/C polymorphism in the gene encoding GYPA; or

one or more polymorphisms which are in linkage disequilibrium with said one or more polymorphisms.

In one embodiment, the data is input by a representative of a healthcare provider.

In another embodiment, the data is input by the subject, their medical advisor or other representative.

Preferably, said system is accessible via the internet or by personal computer.

Preferably, said reference genetic database consists of, comprises or includes the results of an COPD-associated genetic analysis selected from one or more of the genetic analyses described herein and/or the Emphagene™-brand COPD test, preferably the results of an analysis of one or more polymorphisms selected from the group comprising of:
−765 C/G in the promoter of the gene encoding Cyclooxygenase 2 (COX2);
105 C/A in the gene encoding Interleukinl8 (IL18);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tumour Necrosis Factor α (TNFα);
C89Y A/G in the gene encoding SMAD3;
E 469 K A/G in the gene encoding Intracellular Adhesion molecule 1 (ICAM1);
Gly 881 Arg G/C in the gene encoding Caspase (NOD2);
161 G/A in the gene encoding Mannose binding lectin 2 (MBL2);
−1903 G/A in the gene encoding Chymase 1 (CMA1);
Arg 197 Gln G/A in the gene encoding N-Acetyl transferase 2 (NAT2);
−366 G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1 +49 C/T in the gene encoding Elafin; or
−1607 1G/2G in the promoter of the gene encoding Matrix Metalloproteinase 1 (MMP1), with reference to the 1G allele only;
16Arg/Gly in the gene encoding β2 Adrenergic Receptor (ADBR);
130 Arg/Gln (G/A) in the gene encoding Interleukin13 (IL13);
298 Asp/Glu (T/G) in the gene encoding Nitric oxide Synthase 3 (NOS3);
Ile 105 Val (A/G) in the gene encoding Glutathione S Transferase P (GST-P);
Glu 416 Asp (T/G) in the gene encoding Vitamin D binding protein (VDBP);
Lys 420 Thr (A/C) in the gene encoding VDBP;
−1055 C/T in the promoter of the gene encoding IL13;
−308 G/A in the promoter of the gene encoding TNFα;
−511 A/G in the promoter of the gene encoding Interleukin 1B (IL1B);
Tyr 113 His T/C in the gene encoding Microsomal epoxide hydrolase (MEH);
His139 Arg G/A in the gene encoding MEH;
Gln 27 Glu C/G in the gene encoding ADBR;
−1607 1G/2G in the promoter of the gene encoding Matrix Metalloproteinase 1 (MMP1) with reference to the 2G allel only;
−1562 C/T in the promoter of the gene encoding Metalloproteinase 9 (MMP9);
M1 (GSTM1) null in the gene encoding Glutathione S Transferase 1 (GST-1);
1237 G/A in the 3′ region of the gene encoding α1-antitrypsin;
−82 A/G in the promoter of the gene encoding MMP12;
T→C within codon 10 of the gene encoding TGFβ;
760 C/G in the gene encoding SOD3;
−1296 T/C within the promoter of the gene encoding TIMP3; or

the S mutation in the gene encoding α1-antitrypsin.; or

one or more polymorphisms which are in linkage disequilibrium with said one or more polymorphisms.

More preferably, said reference genetic database consists of, comprises or includes the results of all of the genetic analyses described herein and the Emphagene™-brand COPD test.

The present invention further provides a computer program for use in a computer system as described, data files comprising the results of one or more of the genetic analyses described herein or comprising a reference genetic database consisting of, comprising or including the results of one or more of the genetic analyses described herein, and the use of the results of such systems and programs in the determination of a subject's risk of developing COPD, emphysema, or both COPD and emphysema, or in determining the suitability of a subject for an intervention as described herein.

In one embodiment the at least one genetic analysis is the Emphagene™-brand pulmonary test. As used herein, the Emphagene™-brand pulmonary test comprises the methods of determining a subject's predisposition to and/or potential risk of developing chronic obstructive pulmonary disease (COPD) and/or emphysema and related methods as defined in New Zealand Patent Applications No. 539934, No. 541935, No. 545283, and PCT International Application PCT/NZ2006/000103 (published as WO2006/121351) each incorporated herein in its entirety.

In particular, the Emphagene™-brand pulmonary test includes a method of determining a subject's risk of developing one or more obstructive lung diseases comprising analysing a sample from said subject for the presence or absence of one or more polymorphisms selected from the group comprising of:
−765 C/G in the promoter of the gene encoding Cyclooxygenase 2 (COX2);
105 C/A in the gene encoding Interleukin18 (IL18);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tumour Necrosis Factor α (TNFα);
C89Y A/G in the gene encoding SMAD3;
E 469 K A/G in the gene encoding Intracellular Adhesion molecule 1 (ICAM1);
Gly 881Arg G/C in the gene encoding Caspase (NOD2);
161 G/A in the gene encoding Mannose binding lectin 2 (MBL2);
−1903 G/A in the gene encoding Chymase 1 (CMA1);
Arg 197 Gln G/A in the gene encoding N-Acetyl transferase 2 (NAT2);
−366 G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1 +49 C/T in the gene encoding Elafin; or
−1607 1G/2G in the promoter of the gene encoding Matrix Metalloproteinase 1 (MMP1), with reference to the 1G allele only;

wherein the presence or absence of one or more of said polymorphisms is indicative of the subject's risk of developing one or more obstructive lung diseases selected from the group consisting of chronic obstructive pulmonary disease (COPD), emphysema, or both COPD and emphysema.

The methods of the invention can be used to determine the suitability of any subject for an intervention in respect of COPD or emphysema, and to identify those genetic polymorphisms of most use in determining a subject's risk of developing COPD or emphysema.

The predictive methods of the invention allow a number of therapeutic interventions and/or treatment regimens to be assessed for suitability and implemented for a given subject. The simplest of these can be the provision to the subject of motivation to implement a lifestyle change, for example, where the subject is a current smoker, the methods of the invention can provide motivation to quit smoking.

The manner of therapeutic intervention or treatment will be predicated by the nature of the polymorphism(s) and the biological effect of said polymorphism(s). For example, where a susceptibility polymorphism is associated with a change in the expression of a gene, intervention or treatment is preferably directed to the restoration of normal expression of said gene, by, for example, administration of an agent capable of modulating the expression of said gene. Where a SNP allele or genotype is associated with decreased expression of a gene, therapy can involve administration of an agent capable of increasing the expression of said gene, and conversely, where a SNP allele or genotype is associated with increased expression of a gene, therapy can involve administration of an agent capable of decreasing the expression of said gene. Methods useful for the modulation of gene expression are well known in the art. For example, in situations were a SNP allele or genotype is associated with upregulated expression of a gene, therapy utilising, for example, RNAi or antisense methodologies can be implemented to decrease the abundance of mRNA and so decrease the expression of said gene. Alternatively, therapy can involve methods directed to, for example, modulating the activity of the product of said gene, thereby compensating for the abnormal expression of said gene.

Where a susceptibility SNP allele or genotype is associated with decreased gene product function or decreased levels of expression of a gene product, therapeutic intervention or treatment can involve augmenting or replacing of said function, or supplementing the amount of gene product within the subject for example, by administration of said gene product or a functional analogue thereof. For example, where a SNP allele or genotype is associated with decreased enzyme function, therapy can involve administration of active enzyme or an enzyme analogue to the subject. Similarly, where a SNP allele or genotype is associated with increased gene product function, therapeutic intervention or treatment can involve reduction of said function, for example, by administration of an inhibitor of said gene product or an agent capable of decreasing the level of said gene product in the subject. For example, where a SNP allele or genotype is associated with increased enzyme function, therapy can involve administration of an enzyme inhibitor to the subject.

Likewise, when a beneficial (protective) SNP is associated with upregulation of a particular gene or expression of an enzyme or other protein, therapies can be directed to mimic such upregulation or expression in an individual lacking the resistive genotype, and/or delivery of such enzyme or other protein to such individual Further, when a protective SNP is associated with downregulation of a particular gene, or with diminished or eliminated expression of an enzyme or other protein, desirable therapies can be directed to mimicking such conditions in an individual that lacks the protective genotype.

The relationship between the various polymorphisms identified above and the susceptibility (or otherwise) of a subject to COPD, emphysema, or both COPD and emphysema also has application in the design and/or screening of candidate therapeutics. This is particularly the case where the association between a susceptibility or protective polymorphism is manifested by either an upregulation or downregulation of expression of a gene. In such instances, the effect of a candidate therapeutic on such upregulation or downregulation is readily detectable.

For example, in one embodiment existing human lung organ and cell cultures are screened for SNP genotypes as set forth above. (For information on human lung organ and cell cultures, see, e.g.: Bohinski et al. (1996) Molecular and Cellular Biology 14:5671-5681; Collettsolberg et al. (1996) Pediatric Research 39:504; Hermanns et al. (2004) Laboratory Investigation 84:736-752; Hume et al. (1996) In Vitro Cellular & Developmental Biology-Animal 32:24-29; Leonardi et al. (1995) 38:352-355; Notingher et al. (2003) Biopolymers (Biospectroscopy) 72:230-240; Ohga et al. (1996) Biochemical and Biophysical Research Communications 228:391-396; each of which is hereby incorporated by reference in its entirety.) Cultures representing susceptible and protective genotype groups are selected, together with cultures which are putatively “normal” in terms of the expression of a gene which is either upregulated or downregulated where a protective polymorphism is present.

Samples of such cultures are exposed to a library of candidate therapeutic compounds and screened for any or all of: (a) downregulation of susceptibility genes that are normally upregulated in susceptible genotypes; (b) upregulation of susceptibility genes that are normally downregulated in susceptible genotypes; (c) downregulation of protective genes that are normally downregulated or not expressed (or null forms are expressed) in protective genotypes; and (d) upregulation of protective genes that are normally upregulated in protective genotypes. Compounds are selected for their ability to alter the regulation and/or action of susceptibility genes and/or protective genes in a culture having a susceptible genotype.

Similarly, where the polymorphism is one which when present results in a physiologically active concentration of an expressed gene product outside of the normal range for a subject (adjusted for age and sex), and where there is an available prophylactic or therapeutic approach to restoring levels of that expressed gene product to within the normal range, individual subjects can be screened to determine the likelihood of their benefiting from that restorative approach. Such screening involves detecting the presence or absence of the polymorphism in the subject by any of the methods described herein, with those subjects in which the polymorphism is present being identified as individuals likely to benefit from treatment.

It will be appreciated that it is not intended to limit the invention to the above example only, many variations, which may readily occur to a person skilled in the art, being possible without departing from the scope thereof as defined in the accompanying claims.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples only.

### EXAMPLES

The invention will now be described in more detail, with reference to non-limiting examples.

### Example 1

### Case Association Study

### Subject Recruitment

Subjects of European descent who had smoked a minimum of fifteen pack years and diagnosed by a physician with chronic obstructive pulmonary disease (COPD) were recruited. Subjects met the following criteria: were over 50 years old and had developed symptoms of breathlessness after 40 years of age, had a Forced expiratory volume in one second (FEV1) as a percentage of predicted <70% and a FEV1/FVC ratio (Forced expiratory volume in one second/Forced vital capacity) of <79% (measured using American Thoracic Society criteria). Four hundred and seventy four subjects were recruited, of these 59% were male, the mean FEV1/FVC (±95% confidence limits) was 46%, mean FEV1 as a percentage of predicted was 46%. Mean age, cigarettes per day and pack year history was 66 yrs, 23 cigarettes/day and 47 pack years, respectively. Four hundred and eighty four European subjects who had smoked a minimum of twenty pack years and who had never suffered breathlessness and had not been diagnosed with an obstructive lung disease in the past, in particular childhood asthma or chronic obstructive lung disease, were also studied. This control group was recruited through clubs for the elderly and consisted of 60% male, the mean FEV1/FVC ( 95% CI) was 78%, mean FEV1 as a percentage of predicted was 99%. Mean age, cigarettes per day and pack year history was 65 yrs, 24 cigarettes/day and 40 pack years, respectively.

Using a PCR based method (Sandford et al., 1999), all subjects were genotyped for the α1-antitrypsin mutations (S and Z alleles) and those with the ZZ allele were excluded. The COPD and resistant smoker cohorts were matched for subjects with the MZ genotype (5% in each cohort). 190 European blood donors (smoking status unknown) were recruited consecutively through local blood donor services. Sixty-three percent were men and their mean age was 50 years. On regression analysis, the age difference and pack years difference observed between COPD sufferers and resistant smokers was found not to determine FEV or COPD.

This study shows that polymorphisms found in greater frequency in COPD patients compared to controls (and/or resistant smokers) can reflect an increased susceptibility to the development of impaired lung function and COPD. Similarly, polymorphisms found in greater frequency in resistant smokers compared to susceptible smokers (COPD patients and/or controls) can reflect a protective role.

Summary of characteristics for the COPD patients and resistant smokers

| Parameter | COPD | Control smokers |
|---|---|---|
| Mean (1 SD) | N = 474 | N = 484 |
| % male | 59% | 60% |
| Age (yrs) | 66 (9) | 65 (10) |
| Smoking history | | |
| Current smoking (%) | 40% | 48% |
| Age started (yr) | 17 (3) | 17 (3) |
| Yrs smoked | 42 (11) | 35 (11) |
| Pack years* | 47 (20) | 40 (19) |
| Cigarettes/day | 23 (9) | 24 (11) |
| Yrs since quitting | 9.8 (7.4) | 13.9 (8.1) |
| History of other exposures | | |
| Work dust exposure* | 59% | 47% |
| Work fume exposure | 40% | 38% |
| Asbestos exposure* | 22% | 16% |
| FHx of COPD | 37% | 28% |
| FHx of lung cancer* | 11% | 9% |
| Lung Function | | |
| FEV1 (L)* | 1.25 (0.48) | 2.86 (0.68) |
| FEV1 % predicted* | 46% | 99% |
| FEV1/FVC* | 46% (8) | 78 (7) |
| Spirometric COPD#* | 100% | 0% |

ETS = environmental tobacco smoke,
#According to GOLD 2+ criteria,
*P < 0.05.

### Genotyping Methods

Genomic DNA was extracted from whole blood samples (Maniatis, T., Fritsch, E. F. and Sambrook, J., Molecular Cloning Manual. 1989). Purified genomic DNA was aliquoted (10 ng/ul concentration) into 96 well plates and genotyped on a Sequenom™ system (Sequenom™ Autoflex Mass Spectrometer and Samsung 24 pin nanodispenser) using the following sequences, amplification conditions and methods.

The following conditions were used for the PCR multiplex reaction: final concentrations were for 10× Buffer 15 mM MgCl₂ 1.25×, 25 mM MgCl₂ 1.625 mM, dNTP mix 25 mM 500 uM, primers 4 uM 100 nM, Taq polymerase (Qiagen hot start) 0.15 U/reaction, Genomic DNA 10 ng/ul. Cycling times were 95° C. for 15 min, (5° C. for 15 s, 56° C. 30 s, 72° C. 30 s for 45 cycles with a prolonged extension time of 3 min to finish. Shrimp alkaline phosphotase (SAP) treatment was used (2 ul to 5 ul per PCR reaction) incubated at 35° C. for 30 min and extension reaction (add 2 ul to 7 ul after SAP treatment) with the following volumes per reaction of: water, 0.76 ul; hME 10× termination buffer, 0.2 ul; hME primer (10 uM), 1 ul; MassEXTEND enzyme, 0.04 ul.

TABLE A
Sequenom conditions for genotyping

| SNP_ID | 2nd-PCRP | 1st-PCRP | | |
|---|---|---|---|---|
| rs10115703 | ACGTTGGATGCCTCTTATTTCAGCTGCTGG | ACGTTGGATGAGAGAACTCTGATTCTGGCG | | |
| | [SEQ.ID.NO. 1] | [SEQ.ID.NO. 2] | | |
| rs13181 | ACGTTGGATGCACCAGGAACCGTTTATGGC | ACGTTGGATGAGCAGCTAGAATCAGAGGAG | | |
| | [SEQ.ID.NO. 3] | [SEQ.ID.NO. 4] | | |
| rs1799930 | ACGTTGGATGCCTGCCAAAGAAGAAACACC | ACGTTGGATGACGTCTGCAGGTATGTATTC | | |
| | [SEQ.ID.NO. 5] | [SEQ.ID.NO. 6] | | |
| rs2031920 | ACGTTGGATGGTTCTTAATTCATAGGTTGC | ACGTTGGATGCTTCATTTCTCATCATATTTTC | | |
| | [SEQ.ID.NO. 7] | [SEQ.ID.NO. 8] | | |
| rs4073 | ACGTTGGATGACTGAAGCTCCACAATTTGG | ACGTTGGATGGCCACTCTAGTACTATATCTG | | |
| | [SEQ.ID.NO. 9] | [SEQ.ID.NO. 10] | | |
| rs763110 | ACGTTGGATGAGGCTGCAAACCAGTGGAAC | ACGTTGGATGCTGGGCAAACAATGAAAATG | | |
| | [SEQ.ID.NO. 11] | [SEQ.ID.NO. 12] | | |
| rs16969968 | ACGTTGGATGTCTAGAAACACATTGGAAGC | ACGTTGGATGCACGGACATCATTTTCCTTC | | |
| | [SEQ.ID.NO. 13] | [SEQ.ID.NO. 14] | | |
| rs1051730 | ACGTTGGATGTCAAGGACTATTGGGAGAGC | ACGTTGGATGCAGCAGTTGTACTTGATGTC | | |
| | [SEQ.ID.NO. 15] | [SEQ.ID.NO. 16] | | |
| | | EXT1_ | EXT1_ | |
| SNP_ID | UEP_SEQ | CALL | MASS | EXT1_SEQ |
| rs10115703 | TACTCCTGCCTCTAGGAAAGACCACA | G | 8131.3 | TACTCCTGCCTCTAGGAAAGACCACAC |
| | [SEQ.ID.NO. 17] | | | [SEQ.ID.NO. 25] |
| rs13181 | GCAATCTGCTCTATCCTCT | T | 5977.9 | GCAATCTGCTCTATCCTCTT |
| | [SEQ.ID.NO. 18] | | | [SEQ.ID.NO. 26] |
| rs1799930 | TACTTATTTACGCTTGAACCTC | A | 6932.5 | TACTTATTTACGCTTGAACCTCA |
| | [SEQ.ID.NO. 19] | | | [SEQ.ID.NO. 27] |
| rs2031920 | CTTAATTCATAGGTTGCAATTTT | T | 7315.8 | CTTAATTCATAGGTTGCAATTTTA |
| | [SEQ.ID.NO. 20] | | | [SEQ.ID.NO. 28] |
| rs4073 | CACAATTTGGTGAATTATCAA | A | 6716.4 | CACAATTTGGTGAATTATCAAT |
| | [SEQ.ID.NO. 21] | | | [SEQ.ID.NO. 29] |
| rs763110 | AACCCACAGAGCTGCTTTGTATTTC | T | 7863.2 | AACCCACAGAGCTGCTTTGTATTTCA |
| | [SEQ.ID.NO. 22] | | | [SEQ.ID.NO. 30] |
| rs16969968 | CATTGGAAGCTGCGCTC | | | |
| | [SEQ.ID.NO. 23] | | | |
| rs1051730 | TCATCAAAGCCCCAGGCTA | | | |
| | [SEQ.ID.NO. 24] | | | |

| | EXT2 | EXT2 | |
|---|---|---|---|
| SNP_ID | CALL | MASS | EXT2_SEQ |
| rs10115703 | A | 8211.2 | TACTCCTGCCTCTAGGAAAGACCACAT |
| | | | [SEQ.ID.NO. 31] |
| rs13181 | | 6292.1 | GCAATCTGCTCTATCCTCTGC |
| | | | [SEQ.ID.NO. 32] |
| rs1799930 | | 7261.8 | TACTTATTTACGCTTGAACCTCGA |
| | | | [SEQ.ID.NO. 33] |
| rs2031920 | | 7636 | CTTAATTCATAGGTTGCAATTTTGT |
| | | | [SEQ.ID.NO. 34] |
| rs4073 | | 7029.6 | CACAATTTGGTGAATTATCAAAT |
| | | | [SEQ.ID.NO. 35] |
| rs763110 | C | 7879.2 | AACCCACAGAGCTGCTTTGTATTTCG |
| | | | [SEQ.ID.NO. 36] |

### Typing of the HHIP and GYPA SNPs

These SNPs were typed using the Applied Biosystems 7900HT Fast Real-Time PCR System, using genomic DNA extracted from white blood cells and diluted to a concentration of 10 ng/μL, containing no PCR inhibitors, and having an A260/280 ratio greater than 1.7. The reaction mix for each assay was first prepared according to the following table. Enough reaction mix was made to account for all No Template Controls (NTCs) and samples with a surplus 10% to account for pipetting losses. All solutions were kept on ice for the duration of the experiment.

### Reaction Mix

| | Volume (μl) | |
|---|---|---|
| Reagent | One Reaction | n Reactions |
| TaqMan Genotyping Master Mix (2x) | 2.50 | n × 2.50 + 10% |
| SNP Genotyping Assay Mix (40x) | 0.125 | n × 0.125 + 10% |
| DNase-free water | 1.375 | n × 1.375 + 10% |
| Total Volume | 4.00 | |

The reaction plate was then prepared. First, 1 μL of the NTC (DNase-free water) and DNA samples were pipetted into the appropriate wells of the 384-well reaction plate. Each reaction mix was inverted and spun down to mix, and then 4 μL of the reaction mix was added to the appropriate wells of the reaction plate. The reaction plate was then covered with an optical adhesive cover and then briefly centrifuged to spin down contents and eliminate air bubbles. Once preparation of the reaction plate was complete the plate was kept on ice and covered with aluminium foil to protect from the light until it is loaded into the 7900HT Real-Time PCR System.

Sequences were designed commercially by ABI according to the following sequences:

Rs2202507 (GYPA):
[SEQ.ID.NO: 37]
AGACGACACTAGTTTTTAAAGTTTT[G/T]ATTAATCGCTGCTGTGAAGC
TGCAT
Rs1489759 (HHIP):
[SEQ.ID.NO: 38]
GAAATTGTTTTCTTTGGACAACTTG[A/G]CAAAAACCAATCATCTGTCA
GTGAT

After the plate was pre-read with the allelic discrimination document, the amplification run was completed (whether using the 7900HT Real-Time PCR System or another thermal cycler), and after the allelic discrimination post-read was completed the plate was analysed. Automatic calls made by the allelic discrimination document were reviewed using the AQ curve data. The allele calls made on the genotypes were then converted into genotypes.

### Results

The following tables show the results of univariate analysis of the polymorphisms described herein.

TABLE 1
Cerberus 1 (rs 10115703) polymorphism allele and genotype frequencies
in the COPD patients and healthy smoking smokers.

| | Allele* | | Genotype | | |
|---|---|---|---|---|---|
| Cohort | G | A | GG | GA | AA |
| Smoking controls | 878 | 66 | 413 | 52 | 7 |
| n = 472 (%) | (93%) | (7%) | (88%) | (11%) | (2%) |
| COPD n = 705 | 1392 | 118 | 591 | 110 | 4 |
| (%) | (92%) | (8%) | (84%) | (16%) | (1%) |

*number of chromosomes (2n) Genotype
Genotype: GA/AA vs GG in COPD patients compared to smoking controls, OR = 1.4 95% CI 1.0-2.0, χ2 = 3.98, P = 0.05.
GA/AA = susceptible

TABLE 2
XPD (ERCC2) (rs 13181) polymorphism allele and genotype frequencies
in the COPD patients and healthy smoking smokers.

| | Allele* | | Genotype | | |
|---|---|---|---|---|---|
| Cohort | T | G | TT | TG | GG |
| Smoking controls | 539 | 377 | 162 | 215 | 81 |
| n = 458 (%) | (59%) | (41%) | (35%) | (47%) | (18%) |
| COPD n = 698 | 907 | 489 | 295 | 317 | 86 |
| (%) | (65%) | (35%) | (42%) | (45%) | (12%) |

*number of chromosomes (2n) Genotype
Genotype. GG vs TG/TT in COPD patients compared to smoking controls, OR = 0.65 95% CI 0.46-0.920, χ2 = 6.43, P = 0.01.
GG = protective

TABLE 3
NAT2 (rs 1799930) polymorphism allele and genotype frequencies
in the COPD patients and healthy smoking smokers

| | Allele* | | Genotype | | |
|---|---|---|---|---|---|
| Cohort | G | A | GG | GA | AA |
| Smoking controls | 653 | 297 | 222 | 209 | 44 |
| n = 475 (%) | (69%) | (31%) | (47%) | (44%) | (9%) |
| COPD n = 704 | 1018 | 390 | 370 | 278 | 56 |
| (%) | (72%) | (28%) | (53%) | (40%) | (8%) |

*number of chromosomes (2n) Genotype
Genotype. GG vs GA/AA in COPD patients compared to smoking controls, OR = 1.3 95% CI 1.0-1.6, χ2 = 3.84, P = 0.05.
GG genotype = susceptible

TABLE 4
CYP2E1 (rs 2031920) polymorphism allele and genotype frequencies
in the COPD patients and healthy smoking smokers.

| | Allele* | | Genotype | | |
|---|---|---|---|---|---|
| Cohort | C | T | CC | CT | TT |
| Smoking controls | 940 | 14 | 463 | 14 | 0 |
| n = 477 (%) | (99%) | (1%) | (97%) | (3%) | (0%) |
| COPD n = 699 | 1364 | 34 | 665 | 34 | 0 |
| (%) | (98%) | (2%) | (95%) | (5%) | (0%) |

*number of chromosomes (2n) Genotype
Genotype. CT/TT vs CC in COPD patients compared to smoking controls, OR = 1.7 95% CI 0.9-3.3, χ2 = 2.69, P = 0.10.
CT/TT genotype = susceptible

TABLE 5
IL-8 (rs 4073) polymorphism allele and genotype frequencies in the
COPD patients and healthy smoking smokers

| | Allele* | | Genotype | | |
|---|---|---|---|---|---|
| Cohort | T | A | TT | TA | AA |
| Smoking controls | 484 | 468 | 109 | 266 | 101 |
| n = 476 (%) | (51%) | (49%) | (23%) | (56%) | (21%) |
| COPD n = 701 | 780 | 622 | 218 | 344 | 139 |
| (%) | (56%) | (44%) | (31%) | (49%) | (20%) |

*number of chromosomes (2n) Genotype
Genotype. TT vs TA/AA in COPD patients compared to smoking controls, OR = 1.5 95% CI 1.2-2.0, χ2 = 9.49, P = 0.002.
TT genotype = susceptible
Allele. T vs A in COPD patients compared to smoking controls, OR = 1.2 95% CI 1.0-1.4, χ2 = 5.24, P = 0.02.
T allele = susceptible

TABLE 6
α1 anti-chymotrypsin (rs 4934) polymorphism allele and genotype
frequencies in the COPD patients and healthy smoking smokers.

| | Allele* | | Genotype | | |
|---|---|---|---|---|---|
| Cohort | A | G | AA | AG | GG |
| Smoking controls | 503 | 455 | 120 | 263 | 96 |
| n = 479 (%) | (53%) | (47%) | (25%) | (55%) | (20%) |
| COPD n = 698 | 704 | 692 | 180 | 344 | 174 |
| (%) | (50%) | (50%) | (26%) | (49%) | (25%) |

*number of chromosomes (2n) Genotype
Genotype. GG vs AG/AA in COPD patients compared to smoking controls, OR = 1.3 95% CI 1.0-1.8, χ2 = 3.83, P = 0.05.
GG genotype = susceptible

TABLE 7
FasL (rs 763110) polymorphism allele and genotype
frequencies in the COPD patients and healthy smoking smokers.

| | Allele* | | Genotype | | |
|---|---|---|---|---|---|
| Cohort | C | T | CC | CT | TT |
| Smoking controls | 591 | 371 | 188 | 215 | 78 |
| n = 481 (%) | (61%) | (39%) | (39%) | (45%) | (16%) |
| COPD n = 704 | 896 | 512 | 283 | 330 | 91 |
| (%) | (64%) | (36%) | (40%) | (47%) | (13%) |

*number of chromosomes (2n) Genotype
Genotype. TT vs CC/CT in COPD patients compared to smoking controls, OR = 0.8 95% CI 0.6-1.1, χ2 = 2.53, P = 0.11.
TT genotype = protective

TABLE 8
α5 nAChR (rs 16969968) polymorphism allele and genotype
frequencies in the COPD patients and healthy smoking smokers.

| | Allele* | | Genotype | | |
|---|---|---|---|---|---|
| Cohort | G | A | GG | GA | AA |
| Smoking controls | 655 | 295 | 225 | 205 | 45 |
| n = 475 (%) | (69%) | (31%) | (47%) | (43%) | (9%) |
| COPD n = 445 | 551 | 339 | 166 | 219 | 60 |
| (%) | (62%) | (38%) | (37%) | (49%) | (14%) |

*number of chromosomes (2n) Genotype
Genotype. AA vs GG/GA in COPD patients compared to smoking controls, OR = 1.5 95% CI 1.0-2.3, χ2 = 3.65, P = 0.06.
AA genotype = susceptible
Allele. A vs G in COPD patients compared to smoking controls, OR = 1.4 95% CI 1.1-1.7, χ2 = 10.1, P = 0.002.
A allele = susceptible

TABLE 9
nAChR rs1051730 C/T polymorphism allele and genotype frequencies
in control smokers and those with COPD (GOLD ≧2 criteria)

| | Allele* | | Genotype | | |
|---|---|---|---|---|---|
| Cohort | C | T | CC | CT | TT |
| Control smokers | 659 | 293 | 227 | 205 | 44 |
| N = 476 | (69%) | (31%) | 48% | 43% | 9% |
| COPD | 554 | 344 | 168 | 218 | 63 |
| N = 449 | (62%) | (38%) | (37%) | (49%) | (16%) |

*number of chromosomes (2n) Genotype
Genotype. TT vs CC/CT in COPD patients compared to smoking controls, OR = 1.6, 95% CI 1.0-2.5, γ² = 5.2, P = 0.02.
TT = susceptible genotype for COPD.
Allele. T vs C in COPD patients compared to smoking controls, OR = 1.4, 95% CI 1.2-1.7, γ² = 11.6, P = 0.0007.
T = susceptible allele for COPD.

It is noted that the rs16969968 SNP is in linkage disequilibrium with the rs 1051730 and has been estimated to be about 11 kb apart. When the GG, GA and AA genotypes at the rs16969968 polymorphism from each subject (from the combined cohort of controls and COPD patients, n=921) is compared with their rs1051730 SNP genotypes (CC, CT, TT), they are in nearly complete concordance of 99.9% (920/921). This means that in a risk assessment for COPD, either SNP could be used in a panel of SNPs because they are effectively interchangeable and confer the same level of risk (see above). The small statistical variations observed (for example, in Odd's ratio) is due to slightly different numbers in each group.

TABLE 10
HHIP rs1489759 A/G polymorphism allele and genotype frequencies
in control smokers and those with COPD (GOLD ≧2 criteria)

| | Allele* | | Genotype | | |
|---|---|---|---|---|---|
| Cohort | A | G | AA | AG | GG |
| Control smokers | 579 | 389 | 178 | 223 | 83 |
| N = 484 | (60%) | (40%) | (37%) | (46%) | (17%) |
| COPD | 594 | 320 | 187 | 220 | 50 |
| N = 457 | (65%) | (35%) | (41%) | (48%) | (11%) |

*number of chromosomes (2n) Genotype
Genotype: the GG genotype at the HHIP rs1489759 A/G polymorphism is reduced in those with COPD compared to control smokers (11% vs 17%, respectively; OR = 0.59 (95% confidence interval 0.40-0.90), γ2 = 7.46, P = 0.006).
GG = protective genotype for COPD)
Allele: the G allele of the HHIP rs1489759 A/G polymorphism is reduced in those with COPD compared to control smokers (35% vs 40%, respectively; OR = 0.80 (95% confidence interval 0.66-0.97), γ2 = 5.36, P = 0.02).
G = protective allele for COPD)

TABLE 11
GYPA rs2202507 A/C polymorphism allele and genotype frequencies
in control smokers and those with COPD (GOLD ≧2 criteria)

| | Allele* | | Genotype | | |
|---|---|---|---|---|---|
| Cohort | A | C | AA | AC | CC |
| Control smokers | 489 | 471 | 138 | 213 | 129 |
| N = 480 | (51%) | (49%) | (29%) | (44%) | (27%) |
| COPD | 505 | 409 | 136 | 233 | 88 |
| N = 457 | (55%) | (45%) | (30%) | (51%) | (19%) |

*number of chromosomes (2n) Genotype
Genotype: the CC genotype of the GYPA rs2202507 A/C polymorphism is reduced in those with COPD compared to control smokers (19% vs 27%, respectively; OR = 0.65 (95% confidence interval 0.47-0.89), γ² = 7.63, P = 0.006).
CC = protective genotype for COPD
Allele: the C allele of the GYPA rs2202507 A/C polymorphism is reduced in those with COPD compared to control smokers (45% vs 49%, respectively; OR = 0.84 (95% confidence interval 0.70-1.00), γ² = 3.5, P = 0.06).
C = protective allele for COPD

### Example 2

This example presents a combined analysis using a 3 SNP panel comprising the nAChR s16969968 G/A polymorphism, the HHIP rs1489759 A/G polymorphism, and the GYPA rs2202507 A/C polymorphism. Genotype type data for many SNPs can be combined according to a simple algorithm where the presence of the susceptibility genotype (for susceptibility SNPs) scores +1 while the presence of the protective genotype (for protective SNPs) scores −1. This allows geneotype data for a panel of SNPs to be combined to generate a score indicating a level of susceptibility.

Using this approach in the COPD case control study populations described above, the distribution of the combined score using the 3 SNP panel is shown below in Table 12.

TABLE 12
COPD susceptibility score from the 3 SNP panel

| | Low risk score | | Neutral | High risk score |
|---|---|---|---|---|
| Score | −2 | −1 | 0 | 1 |
| Controls | 58 | 100 | 312 | 13 |
| | (12%) | (21%) | (65%) | (3%) |
| COPD | 35 | 60 | 317 | 46 |
| | (8%) | (13%) | (70%) | (10%) |

The frequency of high risk scores and low risk scores in COPD patients compared to controls was 10% vs 3% (high risk) and 21% vs 33% (low risk), respectively, with OR=5.9 (95% confidence interval of 2.9-12.1), γ²=31.45, P<0.0001.

The frequency of high risk+ neutral scores and low risk scores in COPD patients compared to controls was 80% vs 68% (high risk) and 21% vs 33% (low risk), respectively, with OR=1.9 (95% confidence interval of 1.4-2.5), γ²=17.12, P<0.0001.

These data confirm that the combined presence of susceptibility genotypes and absence of protective genotypes is associated with an elevated risk for COPD.

### Example 3

This example presents a combined analysis again using a 3 SNP panel comprising the HHIP rs 1489759 A/G polymorphism, and the GYPA rs2202507 A/C polymorphism, but wherein the nAChR s16969968 G/A polymorphism used in Example 2 has been substituted for the rs1051730 polymorphim. This example illustrates that with the high concordance between these two nAChR SNPs, it is possible to substitute the former SNP with the latter and, using the same approach as described in Example 2 above, derive equivalent risk assessments. The distribution of the combined score using the nAChR rs1051730 C/T polymorphism, the HHIP rs1489759 A/G polymorphism and the GYPA rs2202507 A/C polymorphism is shown below.

TABLE 13
COPD susceptibility score from the substituted 3 SNP panel

| | Low risk score | | Neutral | High risk score |
|---|---|---|---|---|
| Score | −2 | −1 | 0 | 1 |
| Controls | 58 | 100 | 312 | 13 |
| | (12%) | (21%) | (65%) | (3%) |
| COPD | 35 | 60 | 316 | 47 |
| | (8%) | (13%) | (69%) | (10%) |

The frequency of high risk scores and low risk scores in COPD patients compared to controls was 10% vs 3% (high risk) and 21% vs 33% (low risk) respectively with OR=6.0 (95% confidence interval of 3.0-12.4 γ²=32.44, P<0.0001.

The frequency of high risk+ neutral scores and low risk scores in COPD patients compared to controls was 80% vs 68% (high risk) and 21% vs 33% (low risk) respectively with OR=1.9 (95% confidence interval of 1.4-2.5, γ²=17.12, P<0.0001.

These data confirm that the substitution of one SNP with another in LD has no effect on the risk assessment and confirms that SNPs in LD (with similar gene frequencies and high concordance on genotyping) can be used as alternative markers in risk assessment.

Allele frequency data on a further example of a SNP in LD suitable for substitution with either the rs16969968 polymorphism or the rs1051730 polymorphism in the nAChR gene is presented in Table 14 below.

TABLE 14
Allele frequency data for nAChR polymorphisms and a SNP in LD

| | Major | Minor | Position | Closest Gene |
|---|---|---|---|---|
| rs8034191 | T | C | 76,593,078 | LOC123688 |
| | 0.567 | 0.433 | | (hypothetical) |
| rs16969968 | G | A | 76,669,980 | CHRNA5 |
| | 0.576 | 0.424 | | |
| rs1051730 | C | T | 76,681,394 | CHRNA3 |
| | 0.57 | 0.43 | | |

Chr15: 76580000 . . . 76710000

As shown in FIG. 1, the HapMap database reports the 3 SNPs depicted in Table 14 are in complete LD (D′=1.0).

### Example 4

Table 15 below presents representative examples of polymorphisms in linkage disequilibrium with the polymorphisms specified herein. Examples of such polymorphisms can be located using public databases, such as that available at www.hapmap.org. Specified polymorphisms are shown in bold and parentheses. The rs numbers provided are identifiers unique to each polymorphism.

TABLE 15
Polymorphism reported to be in LD with polymorphisms specified herein.
CER1

| | | | | |
|---|---|---|---|---|
| rs10810224 | rs17289263 | rs3761666 | rs13286013 | rs7022304 |
| rs7870750 | rs10961679 | rs7022400 | rs10121506 | rs10961680 |
| rs11999277 | rs10118242 | rs10961681 | rs1494360 | rs10118290 |
| rs951273 | rs1494359 | rs16932212 | rs2131883 | rs1494358 |
| rs11794846 | rs2131882 | rs1494357 | rs10122395 | rs12338263 |
| rs3747532 | rs10125285 | rs12338303 | (rs10115703) | rs1494351 |
| rs12338380 | rs10122490 | rs1494350 | rs2088042 | rs7018937 |
| rs10961683 | rs12347640 | rs12115314 | rs10961684 | rs10122817 |
| rs7035643 | rs11793334 | rs12115487 | rs10961682 | rs7019731 |
| rs11789968 | rs7019387 | rs10810225 | rs3761665 | rs3819004 |
| rs10123442 | rs7036635 | rs10810226 | | |

XPD, ERCC2

| | | | | |
|---|---|---|---|---|
| rs1799793 | rs238409 | rs3916858 | rs3916876 | rs7257638 |
| rs3916838 | rs106433 | rs238417 | rs3916816 | rs50871 |
| rs3916860 | rs3916878 | rs3916817 | rs50872 | rs3916861 |
| rs3916879 | rs3916818 | rs3916839 | rs3916862 | rs1799787 |
| rs3916819 | rs3916840 | rs3916863 | rs1799788 | rs3916820 |
| rs3916841 | rs238412 | rs1799789 | rs238404 | rs3916842 |
| rs3916864 | rs16979773 | rs3916821 | rs3916843 | rs11668936 |
| rs1052555 | rs3916822 | rs3916844 | rs3916866 | rs3916881 |
| rs238403 | rs7251321 | rs2070831 | rs3916882 | rs171140 |
| rs3916845 | rs3916868 | rs3916883 | rs3895625 | rs3916846 |
| rs238413 | rs238418 | rs3916824 | rs3916847 | rs238414 |
| rs3916885 | rs3916825 | rs3916848 | rs3916870 | rs3916886 |
| rs3916826 | rs238410 | rs3916871 | rs1799790 | rs3916827 |
| rs238411 | rs3916872 | (rs13181 - 751 G/T) | rs3916830 | rs3916849 |
| rs238415 | rs3916831 | rs3916850 | rs3916873 | rs3916832 |
| rs3916851 | rs3932979 | rs3916833 | rs3916853 | rs238416 |
| rs3916834 | rs3916854 | rs3916874 | rs3916835 | rs3916855 |
| rs11667568 | rs3916836 | rs3916856 | rs3916875 | rs3916837 |
| rs3916857 | rs11666730 | | | |

NAT2

| | | | | |
|---|---|---|---|---|
| rs11780272 | rs1495744 | rs2101857 | rs7832071 | rs13363820 |
| rs1805158 | rs6984200 | rs1801279 | rs13277605 | rs1041983 |
| rs9987109 | rs1801280 | rs7820330 | rs4986996 | rs7460995 |
| rs12720065 | rs2087852 | rs4986997 | rs2101684 | rs1799929 |
| rs7011792 | (rs1799930 - | rs1390358 | rs923796 | rs1208 |
| | Arg 197 Gln) | | | |
| rs4546703 | rs1799931 | rs4634684 | rs2552 | rs2410556 |
| rs4646247 | rs11996129 | rs971473 | rs4621844 | rs721398 |
| rs11785247 | rs1115783 | rs1115784 | rs1961456 | rs1112005 |
| rs11782802 | rs973874 | | | |

CYP2E1

| | | | | |
|---|---|---|---|---|
| rs7091961 | rs12776213 | rs1329148 | rs10857736 | rs12262150 |
| rs6537611 | rs10857732 | rs10857737 | rs9418989 | rs6537612 |
| rs10857733 | rs12776473 | rs10776686 | rs10466129 | rs11101801 |
| rs10466130 | rs4838767 | rs11101810 | rs9419081 | rs9418990 |
| rs9419082 | rs10857738 | rs11101803 | rs11101811 | rs10776687 |
| rs3813865 | rs4838688 | rs3813866 | rs11101805 | rs11575869 |
| rs2031918 | rs8192766 | rs2031919 | rs11575870 | rs11101806 |
| rs6413423 | rs4838689 | (rs3813867 - | rs10857734 | rs4838768 |
| | | 1019 G/C Pst1) | | |
| rs6413422 | rs11101807 | (rs2031920 - | rs11101808 | rs11101809 |
| | | Rsa1 C/T) | | |
| rs10857735 | | | | |

IL8

| | | | | |
|---|---|---|---|---|
| rs4694635 | rs2227527 | rs2227543 | rs11730560 | rs11730284 |
| rs1957663 | rs7682639 | rs12420 | rs13106097 | rs11944402 |
| rs4694636 | rs2227529 | rs16849942 | rs4694178 | rs7658422 |
| rs16849925 | rs2227530 | rs3181685 | rs4694637 | rs11940656 |
| rs16849928 | rs2227531 | rs11733933 | rs11729759 | rs1951700 |
| rs11730667 | rs2227532 | rs2227544 | rs10938093 | rs1951699 |
| rs16849934 | rs2227534 | rs2227545 | rs13109377 | rs1957662 |
| (rs4073 - 251 A/T) | rs2227550 | rs1951236 | rs16849938 | rs2227546 |
| rs1951237 | rs6831816 | rs2227535 | rs1126647 | rs6446955 |
| rs2227517 | rs2227536 | rs11545234 | rs6446956 | rs2227518 |
| rs2227537 | rs2227548 | rs6446957 | rs2227519 | rs2227538 |
| rs10938092 | rs16849945 | rs2227520 | rs1803205 | rs13112910 |
| rs1951239 | rs2227521 | rs2227539 | rs13142454 | rs1951240 |
| rs2227522 | rs3756069 | rs11937527 | rs1957661 | rs2227523 |
| rs2227307 | rs12647924 | rs7674884 | rs2227524 | rs2227549 |
| rs13152254 | rs16849958 | rs2227525 | rs2227540 | rs13138765 |
| rs17202249 | rs2227526 | rs2227306 | rs13139170 | rs1951242 |

FasL

| | | | | | |
|---|---|---|---|---|---|
| rs1894626 | rs2859235 | rs2639617 | rs3021335 | rs16844867 | rs2639622 |
| rs10912122 | rs2859239 | rs2933547 | rs9787393 | rs2639621 | rs2639618 |
| rs2639616 | rs2859244 | rs9787248 | rs2859228 | rs2859236 | rs2131373 |
| rs2859245 | rs12080307 | rs2859229 | rs10798130 | rs12130118 | rs10753023 |
| rs749154 | rs1492899 | rs16844856 | rs2859240 | rs10798133 | rs749155 |
| rs12082528 | rs2021839 | rs2639615 | rs2859246 | (rs763110) | rs4304626 |
| rs2021838 | rs2859241 | rs2859247 | rs2859233 | rs2859237 | rs2859242 |
| rs2639614 | rs2859234 | rs2859238 | rs2859243 | rs2859248 | |

nAChR

| | | | | | |
|---|---|---|---|---|---|
| rs2869030 | rs12909921 | rs11858804 | rs11636131 | rs684513 | rs7178162 |
| rs4887053 | rs12910090 | rs11631834 | rs11637127 | rs7495275 | (rs1051730) |
| rs16969840 | rs12916396 | rs11631892 | rs11632604 | rs7165657 | rs8192481 |
| rs12439399 | rs12916558 | rs7497617 | rs12910289 | rs7166003 | rs3743078 |
| rs4436747 | rs2656071 | rs4887060 | rs7169751 | rs7178897 | rs3743077 |
| rs8043201 | rs2656069 | rs12593550 | rs1504546 | rs1472739 | rs1317286 |
| rs2869032 | rs2656068 | rs8026308 | rs16969931 | rs667282 | rs938682 |
| rs11856232 | rs2568496 | rs11636431 | rs12906951 | rs11636592 | rs12904589 |
| rs4381564 | rs2869048 | rs10450995 | rs3885951 | rs479385 | rs12914385 |
| rs2869045 | rs5020118 | rs10450964 | rs11633027 | rs16969948 | rs11637630 |
| rs2568495 | rs2017512 | rs965604 | rs931794 | rs588765 | rs2869546 |
| rs16969845 | rs2656065 | rs13180 | rs12913194 | rs6495306 | rs7177514 |
| rs2869046 | rs2568485 | rs2292116 | rs7180652 | rs16969949 | rs6495308 |
| rs2568498 | rs2568483 | rs9920411 | rs12916999 | rs12903839 | rs12443170 |
| rs12911087 | rs2656062 | rs2055588 | rs2036534 | rs17486278 | rs8042059 |
| rs2656057 | rs11639224 | rs3743079 | rs7164644 | rs1875869 | rs8042374 |
| rs1394371 | rs905742 | rs8033501 | rs12915366 | rs601079 | rs4887069 |
| rs12101964 | rs905741 | rs1062980 | rs12916483 | rs495956 | rs3743076 |
| rs12903150 | rs1964678 | rs17406522 | rs3813572 | rs680244 | rs3743075 |
| rs2656059 | rs2009746 | rs12441192 | rs3813571 | rs1878398 | rs3743074 |
| rs2656060 | rs2938674 | rs16969906 | rs3813570 | rs621849 | rs3743073 |
| rs2036530 | rs2938673 | rs3417 | rs12901682 | rs569207 | rs8040868 |
| rs12899425 | rs2958720 | rs11637193 | rs4886571 | rs637137 | rs8192475 |
| rs12899131 | rs1394372 | rs12914367 | rs4243083 | rs7180002 | rs1878399 |
| rs2568500 | rs17484235 | rs2055587 | rs2292117 | rs11633585 | rs6495309 |
| rs16969846 | rs17405883 | rs4362358 | rs11551779 | rs8026141 | rs1948 |
| rs2568484 | rs9972290 | rs5019044 | rs11858230 | rs692780 | rs7178270 |
| rs17483548 | rs4886569 | rs7171274 | rs8025429 | rs11637635 | rs3743072 |
| rs2869047 | rs3817092 | rs12906676 | rs4887062 | rs481134 | rs12914008 |
| rs17405217 | rs4299116 | rs6495304 | rs4887063 | rs951266 | rs17487223 |
| rs924840 | rs1504550 | rs7168796 | rs8053 | rs10519205 | rs950776 |
| rs2938671 | rs12591395 | rs16969914 | rs1979907 | rs555018 | |
| rs17483721 | rs12910910 | rs9788682 | rs1979906 | rs647041 | |
| rs2568487 | rs8043227 | rs9788721 | rs1979905 | rs12898919 | |
| rs1847529 | rs7162301 | rs7164594 | rs4887064 | rs12903575 | |
| rs1847528 | rs11634990 | rs16969920 | rs12907966 | rs17408276 | |
| rs8041628 | rs11072766 | rs16969922 | rs1504547 | (rs16969968) | |
| rs11630228 | rs11072767 | (rs8034191) | rs8024878 | rs518425 | |
| rs2568488 | rs17484524 | rs4380026 | rs16969941 | rs11635346 | |
| rs2656053 | rs8026728 | rs12591557 | rs880395 | rs514743 | |
| rs2568491 | rs8042238 | rs10519203 | rs905740 | rs615470 | |
| rs16969858 | rs8042260 | rs12914694 | rs7164030 | rs7163480 | |
| rs2568492 | rs16969892 | rs7163730 | rs8037347 | rs12899226 | |
| rs2656052 | rs8027404 | rs8031948 | rs7183333 | rs660652 | |
| rs2568494 | rs11858961 | rs4461039 | rs4275821 | rs472054 | |
| rs7181486 | rs12903295 | rs1504545 | rs7173512 | rs8029939 | |
| rs2656073 | rs12904234 | rs952215 | rs4887065 | rs578776 | |
| rs17483929 | rs7177092 | rs952216 | rs2036527 | rs6495307 | |
| rs10519198 | rs16969899 | rs12902493 | rs11636732 | rs12910984 | |
| rs2958719 | rs8032410 | rs11544874 | rs2944674 | rs8033506 | |

HHIP

| | | | | | |
|---|---|---|---|---|---|
| rs1032295 | rs2220516 | rs7655625 | rs9685759 | rs1032296 | rs2035743 |
| rs7673529 | rs7677662 | rs1032297 | rs6537292 | rs596165 | rs7700244 |
| rs1512281 | rs13104277 | rs451825 | rs6842331 | rs12504628 | rs10017175 |
| rs12641683 | rs1398243 | rs7697189 | rs6824927 | rs13118928 | rs7666523 |
| rs7681384 | rs12511230 | rs610411 | rs1186270 | rs11943195 | rs10028899 |
| rs12505157 | rs1542726 | rs4835637 | rs17019464 | rs426979 | rs4835638 |
| rs6820700 | rs404618 | rs1489757 | rs1489759 | rs427260 | rs6829956 |
| rs383501 | rs17019485 | rs6854832 | rs386213 | rs6537296 | rs7340879 |
| rs1873297 | rs17019486 | rs11938704 | rs11932233 | rs462044 | rs3891822 |
| rs995759 | rs13140176 | rs1512285 | rs995758 | rs6828255 | rs6821114 |
| rs12509311 | rs1512288 | rs6845536 | rs4834988 | rs6817273 | rs1489762 |
| rs11100860 | rs593918 | rs1489761 | rs11934806 | rs2175586 | rs7692102 |
| rs6842889 | rs389937 | rs7673263 | rs6813222 | rs1473100 | rs7673872 |
| rs389291 | rs17019499 | rs7685166 | rs10519717 | rs13136959 | rs13147758 |
| rs9998537 | rs1844430 | rs13148031 | rs1828591 | rs6537297 | rs7689654 |
| rs6537295 | rs13126322 | rs720484 | rs6840009 | rs423625 | rs720485 |
| rs17019476 | rs13101284 | rs6811415 | rs6810579 | rs10013495 | rs6828540 |
| rs6816405 | rs13141641 | rs13113237 | rs17019477 | rs6852830 | rs2130339 |
| rs12510044 | rs2220548 | rs6830832 | rs457881 | rs12643826 | rs11938745 |
| rs6821908 | rs11724319 | rs6850426 | rs6829350 | rs1996020 | rs394216 |
| rs1489766 | rs11933312 | rs2130338 | rs1980057 | rs7670758 | rs11938808 |
| rs7671897 | rs7691995 | | | | |

GYPA

| | | | | | |
|---|---|---|---|---|---|
| rs13118083 | rs6849200 | rs885439 | rs11100855 | rs6814459 | rs6836202 |
| rs4835177 | rs7654571 | rs749316 | rs4533790 | rs1118190 | rs2657798 |
| rs7676032 | rs13142439 | rs13118515 | rs6856698 | rs989346 | rs4420930 |
| rs12510916 | rs13141892 | rs6828489 | rs6537279 | rs4376087 | rs1490146 |
| rs11100859 | rs13108250 | rs12645006 | rs12500355 | rs17019365 | rs13142879 |
| rs13137424 | rs12641258 | rs4835634 | rs6828795 | rs398962 | rs12640712 |
| rs1857835 | rs7654506 | rs1490147 | rs13149808 | rs6830386 | rs1505772 |
| rs990768 | rs11727645 | rs6825094 | rs12640763 | rs17766287 | rs951848 |
| rs11728562 | rs17712227 | rs7660767 | rs4371571 | rs1394999 | rs11731448 |
| rs1512287 | rs11100850 | rs4371572 | rs17767138 | rs1873296 | rs612550 |
| rs1505771 | rs7688932 | rs2719333 | rs6847170 | rs11722531 | rs7674433 |
| rs7683975 | rs2719332 | rs1490148 | rs461265 | rs4256191 | rs10009317 |
| rs4362772 | rs11940095 | rs13149519 | rs4321584 | rs2657799 | rs13109426 |
| rs4552414 | rs13143949 | rs1876116 | rs6537281 | rs17767210 | rs1505770 |
| rs13143967 | rs11100851 | rs7378179 | rs17019336 | rs1490149 | rs13144144 |
| rs2174527 | rs4240362 | rs17019340 | rs7689824 | rs13116441 | rs6842640 |
| rs6840917 | rs11726621 | rs2657805 | rs7684769 | rs6842885 | rs2657794 |
| rs4290852 | rs17019370 | rs7654708 | rs7655235 | rs4465995 | rs13117231 |
| rs390898 | rs12640256 | rs6836137 | rs986849 | rs13111832 | rs7675095 |
| rs973796 | rs7377575 | rs970022 | rs13135495 | rs2636153 | rs13116963 |
| rs4317155 | rs986241 | rs13135513 | rs13108069 | rs12641251 | rs4031150 |
| rs1505768 | rs13137063 | rs13108077 | rs12639777 | rs2202507 | rs10029738 |
| rs13112056 | rs13113788 | rs1490150 | rs4306911 | rs10029931 | rs13117676 |
| rs13108244 | rs2048536 | rs6537278 | rs7681655 | rs1505762 | rs13108260 |
| rs12500946 | rs10030023 | rs4469023 | rs7675830 | rs438691 | rs8180243 |
| rs2657804 | rs4370082 | rs6537289 | rs443126 | rs11935246 | rs7661046 |
| rs7665807 | rs12512146 | rs625071 | rs6827794 | rs7375701 | rs4318599 |
| rs12499537 | rs438682 | rs1512282 | rs6852276 | rs1394998 | rs17019349 |
| rs423784 | rs10222998 | rs6858668 | rs988599 | rs11932998 | rs397724 |
| rs7671881 | rs13105210 | rs13121032 | rs612176 | rs17019376 | rs7654793 |
| rs2719341 | rs6537284 | rs627063 | rs11733975 | rs11727583 | rs6822064 |
| rs4642189 | rs7695767 | rs2719336 | rs13142776 | rs6840871 | rs4383570 |
| rs7678519 | rs6817612 | rs17019408 | rs2352767 | rs1505765 | rs7678522 |
| rs11735110 | rs7678427 | rs4493485 | rs4501169 | rs11726412 | rs1512289 |
| rs4266245 | rs7693416 | rs2719342 | rs2130499 | rs12503296 | rs7692044 |
| rs1907019 | rs440058 | rs17709487 | rs6811667 | rs2719340 | rs1512279 |
| rs7676787 | rs12645910 | rs2200942 | rs12499011 | rs987246 | rs1505766 |
| rs1602238 | rs17019381 | rs6834183 | rs6537285 | rs13103448 | rs1398245 |
| rs7699261 | rs6537286 | rs12499685 | rs11729536 | rs4292285 | rs4342151 |
| rs17019354 | rs17516 | rs17766168 | rs4610282 | rs2719337 | rs11722105 |

### Discussion

The above results show that several polymorphisms were associated with either susceptibility and/or resistance to obstructive lung disease in those exposed to smoking environments. The associations of individual polymorphisms on their own, while of discriminatory value, are unlikely to offer an acceptable prediction of disease. However, in combination these polymorphisms distinguish susceptible smokers (with COPD) from those who are resistant. The polymorphisms represent both promoter polymorphisms, thought to modify gene expression and hence protein synthesis, and exonic polymorphisms known to alter amino-acid sequence (and likely expression and/or function) in processes known to underlie lung remodelling. The polymorphisms identified here are found in genes encoding proteins central to these processes which include inflammation, matrix remodelling and oxidant stress.

In the comparison of smokers with COPD and matched smokers with near normal lung function, several polymorphisms were identified as being found in significantly greater or lesser frequency than in the comparator groups (including the blood donor cohort).
In the analysis of the rs10115703 G/A polymorphism in the gene encoding Cerberus 1, the GA and AA genotypes were found to be significantly greater in the COPD patients compared to the healthy smoker control cohort (OR=1.4, P=0.05) consistent with a susceptibility role (see Table 1).
In the analysis of the rs13181 G/T polymorphism in the gene encoding xeroderma pigmentosum complementation group D, the GG genotype was found to be significantly greater in the resistant smoker cohort compared to the COPD cohort (OR=0.65, P=0.01) consistent with a protective role (see Table 2).
In the analysis of the rs1799930 G/A polymorphism in the gene encoding N-Acetyl transferase 2, the GG genotype was found to be significantly greater in the COPD cohort compared to the controls (OR=1.3, P=0.05) consistent with a susceptibility role (see Table 3).
In the analysis of the rs2031920 C/T polymorphism in the gene encoding cytochrome P450 2E1, the CT and TT genotypes were found to be significantly greater in the COPD cohort compared to the resistant smoker cohort (OR=1.7, P=0.10) consistent with a susceptibility role (see Table 4).
In the analysis of the rs4073 T/A polymorphism in the gene encoding Interleukin8 (IL-8), the T allele and the TT genotype were found to be greater in the COPD cohort compared to the controls (OR=1.2, P=0.02, and OR=1.5, P=0.002, respectively) consistent with a susceptibility role (see Table 5).
In the analysis of the rs4934 G/A polymorphism in the gene encoding α1 anti-chymotrypsin, the GG genotype was found to be greater in the COPD cohort compared to the controls (OR=1.3, P=0.05) consistent with a susceptibility role (see Table 6).
In the analysis of the rs763110 C/T polymorphism in the gene encoding Fas ligand, the TT genotype was found to be greater in the resistant smoker cohort compared to the COPD cohort (OR=0.8, P=0.11) consistent with a protective role (see Table 7).
In the analysis of the rs16969968 G/A polymorphism in the gene encoding α5 nicotinic acetylcholine receptor subunit, the A allele and the AA genotype were found to be greater in the COPD cohort compared to the controls (OR=1.4, P=0.002, and OR=1.5, P=0.06) consistent with susceptibility roles (see Table 8).
In the analysis of the rs1051730 C/T polymorphism in the gene encoding α5 nicotinic acetylcholine receptor subunit, the T allele and the TT genotype were found to be greater in the COPD cohort compared to the controls (OR=1.4, P=0.0007, and OR=1.6, P=0.02) consistent with susceptibility roles (see Table 9).
In the analysis of the rs1489759 A/G polymorphism in the gene encoding human hedgehog interacting protein, the G allele and the GG genotype were found to be greater in the resistant smoker cohort compared to the COPD cohort (OR=0.8, P=0.02, and OR=0.59, P=0.006) consistent with protective roles (see Table 10).
In the analysis of the rs2202507 A/C polymorphism in the gene encoding glycophorin A, the C allele and the CC genotype were found to be greater in the resistant smoker cohort compared to the COPD cohort (OR=0.84, P=0.06, and OR=0.65, P=0.006) consistent with protective roles (see Table 11).

It is accepted that the disposition to chronic obstructive lung diseases (eg. emphysema and COPD) is the result of the combined effects of the individual's genetic makeup and their lifetime exposure to various aero-pollutants of which smoking is the most common Similarly it is accepted that COPD encompasses several obstructive lung diseases and characterised by impaired expiratory flow rates (eg FEV1). The data herein suggest that several genes can contribute to the development of COPD. A number of genetic mutations working in combination either promoting or protecting the lungs from damage can be involved in elevated resistance or susceptibility.

From the analyses of the individual polymorphisms, 6 susceptibility and 2 protective genotypes were identified and analysed for their frequencies in the smoker cohort consisting of resistant smokers and those with COPD. The frequencies of resistant smokers and smokers with COPD can be compared according to the presence absence of these genotypes.

These findings indicate that the methods of the present invention can be predictive of COPD, emphysema, or both COPD and emphysema in an individual well before symptoms present.

These findings therefore also present opportunities for therapeutic interventions and/or treatment regimens, as discussed herein. Briefly, such interventions or regimens can include the provision to the subject of motivation to implement a lifestyle change, or therapeutic methods directed at normalising aberrant gene expression or gene product function. For example, the A allele at a polymorphic site in gene is associated with increased expression of the gene relative to that observed with the C allele. The C allele is protective with respect to predisposition to or potential risk of developing COPD, emphysema, or both COPD and emphysema, whereby a suitable therapy in subjects known to possess the A allele can be the administration of an agent capable of reducing expression of the gene. An alternative suitable therapy can be the administration to such a subject of a inhibitor of the gene or gene product, such as additional therapeutic approaches, gene therapy, RNAi. In another example, the C allele at a polymorphic site in the promoter of a gene is associated with susceptibility to COPD, emphysema, or both COPD and emphysema. The G allele at the polymorphic site is associated with increased protein levels, whereby a suitable therapy in subjects known to possess the C allele can be the administration of an agent capable of increasing expression of the gene. In still another example, the GG genotype at a polymorphic site in the promoter of a gene is associated with susceptibility to COPD, emphysema, or both COPD and emphysema. The GG allele is reportedly associated with increased binding of a repressor protein and decreased transcription of the gene. A suitable therapy can be the administration of an agent capable of decreasing the level of repressor and/or preventing binding of the repressor, thereby alleviating its downregulatory effect on transcription. An alternative therapy can include gene therapy, for example the introduction of at least one additional copy of the plasminogen activator inhibitor gene having a reduced affinity for repressor binding (for example, a gene copy having a CC genotype at the polymorphic site).

Suitable methods and agents for use in such therapy are well known in the art, and are discussed herein.

The identification of both susceptibility and protective polymorphisms as described herein also provides the opportunity to screen candidate compounds to assess their efficacy in methods of prophylactic and/or therapeutic treatment. Such screening methods involve identifying which of a range of candidate compounds have the ability to reverse or counteract a genotypic or phenotypic effect of a susceptibility polymorphism, or the ability to mimic or replicate a genotypic or phenotypic effect of a protective polymorphism.

Still further, methods for assessing the likely responsiveness of a subject to an available prophylactic or therapeutic approach are provided. Such methods have particular application where the available treatment approach involves restoring the physiologically active concentration of a product of an expressed gene from either an excess or deficit to be within a range which is normal for the age and sex of the subject. In such cases, the method comprises the detection of the presence or absence of a susceptibility polymorphism which when present either upregulates or downregulates expression of the gene such that a state of such excess or deficit is the outcome, with those subjects in which the polymorphism is present being likely responders to treatment.

Examples of polymorphisms in linkage disequilibrium with the polymorphisms specified herein can be located using public databases, such as that available at www.hapmap.org, using, for example a unique identifier such as the rs number.

### INDUSTRIAL APPLICATION

The present invention is directed to methods for assessing a subject's risk of developing chronic obstructive pulmonary disease (COPD), emphysema, or both COPD and emphysema. The methods comprise the analysis of polymorphisms herein shown to be associated with increased or decreased risk of developing COPD, emphysema, or both COPD and emphysema, or the analysis of results obtained from such an analysis. The use of polymorphisms herein shown to be associated with increased or decreased risk of developing COPD, emphysema, or both COPD and emphysema in the assessment of a subject's risk are also provided, as are nucleotide probes and primers, kits, and microarrays suitable for such assessment. Methods of treating subjects having the polymorphisms herein described are also provided. Methods for screening for compounds able to modulate the expression of genes associated with the polymorphisms herein described are also provided.

### REFERENCES

Maniatis, T., Fritsch, E. F. and Sambrook, J., Molecular Cloning Manual. 1989.
Sandford A J, et al., 1999. Z and S mutations of the α1-antitrypsin gene and the risk of chronic obstructive pulmonary disease. Am J Respir Cell Mol Biol. 20; 287-291.

All patents, publications, scientific articles, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of various embodiments or preferred embodiments and are exemplary only and not intended as limitations on the scope of the invention. Other objects, aspects, examples and embodiments will occur to those skilled in the art upon consideration of this specification, and are encompassed within the spirit of the invention as defined by the scope of the claims. It will be readily apparent to one skilled in the art that varying substitutions and modifications can be made to the invention disclosed herein without departing from the scope and spirit of the invention. The invention illustratively described herein suitably can be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of ” may be replaced with either of the other two terms in the specification, thus indicating additional examples, having different scope, of various alternative embodiments of the invention. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of assessing a subject's risk of developing chronic obstructive pulmonary disease, emphysema, or both chronic obstructive pulmonary disease and emphysema, said method comprising:
providing the result of one or more genetic tests of a sample from the subject, and
analysing the result for the presence or absence of one or more polymorphisms selected from the group consisting of:
rs10115703 G/A polymorphism in the gene encoding Cer 1;
rs13181 G/T polymorphism in the gene encoding XPD;
rs1799930 G/A polymorphism in the gene encoding NAT2;
rs2031920 C/T polymorphism in the gene encoding CYP2E1;
rs4073 T/A polymorphism in the gene encoding IL-8;
rs763110 C/T polymorphism in the gene encoding FasL;
rs16969968 G/A polymorphism in the gene encoding α5-nAChR;
rs1051730 C/T polymorphism in the gene encoding α5-nAChR; and
one or more polymorphisms in linkage disequilibrium with one or more of these polymorphisms;
wherein the presence or absence of one or more of said polymorphisms is indicative of the subject's risk of developing chronic obstructive pulmonary disease, emphysema, or both chronic obstructive pulmonary disease and emphysema.

2. The method of claim 1, comprising:
analysing the result for the presence of one or more further polymorphisms selected from the group consisting of:
the rs4934 G/A polymorphism in the gene encoding α1 anti-chymotrypsin;
the rs1489759 A/G polymorphism in the gene encoding HHIP; and
the rs2202507 A/C polymorphism in the gene encoding GYPA.

3. The method according to claim 2, comprising:
analysing the result for the presence or absence of one or more further polymorphisms selected from the group consisting of:
−765 C/G in the promoter of the gene encoding Cyclooxygenase 2 (COX2);
105 C/A in the gene encoding Interleukin18 (IL18);
−133 G/C in the promoter of the gene encoding IL18;
−675 4G/5G in the promoter of the gene encoding Plasminogen Activator Inhibitor 1 (PAI-1);
874 A/T in the gene encoding Interferon-γ (IFN-γ);
+489 G/A in the gene encoding Tumour Necrosis Factor α (TNFα);
C89Y A/G in the gene encoding SMAD3;
E 469 K A/G in the gene encoding Intracellular Adhesion molecule 1 (ICAM1);
Gly 881Arg G/C in the gene encoding Caspase (NOD2);
161 G/A in the gene encoding Mannose binding lectin 2 (MBL2);
−1903 G/A in the gene encoding Chymase 1 (CMA1);
Arg 197 Gln G/A in the gene encoding N-Acetyl transferase 2 (NAT2);
−366 G/A in the gene encoding 5 Lipo-oxygenase (ALOX5);
HOM T2437C in the gene encoding Heat Shock Protein 70 (HSP 70);
+13924 T/A in the gene encoding Chloride Channel Calcium-activated 1 (CLCA1);
−159 C/T in the gene encoding Monocyte differentiation antigen CD-14 (CD-14);
exon 1 +49 C/T in the gene encoding Elafin;
−1607 1G/2G in the promoter of the gene encoding Matrix Metalloproteinase 1 (MMP1), with reference to the 1G allele only;
16Arg/Gly in the gene encoding β2 Adrenergic Receptor (ADBR);
130 Arg/Gln (G/A) in the gene encoding Interleukin13 (IL13);
298 Asp/Glu (T/G) in the gene encoding Nitric oxide Synthase 3 (NOS3);
Ile 105 Val (A/G) in the gene encoding Glutathione S Transferase P (GST-P);
Glu 416 Asp (T/G) in the gene encoding Vitamin D binding protein (VDBP);
Lys 420 Thr (A/C) in the gene encoding VDBP;
−1055 C/T in the promoter of the gene encoding IL13;
−308 G/A in the promoter of the gene encoding TNFα;
−511 A/G in the promoter of the gene encoding Interleukin 1B (IL1B);
Tyr 113 His T/C in the gene encoding Microsomal epoxide hydrolase (MEH);
His139 Arg G/A in the gene encoding MEH;
Gln 27 Glu C/G in the gene encoding ADBR;
−1607 1G/2G in the promoter of the gene encoding Matrix Metalloproteinase 1 (MMP1) with reference to the 2G allele only;
−1562 C/T in the promoter of the gene encoding Metalloproteinase 9 (MMP9);
M1 (GSTM1) null in the gene encoding Glutathione S Transferase 1 (GST-1);
1237 G/A in the 3′ region of the gene encoding α1-antitrypsin;
−82 A/G in the promoter of the gene encoding MMP12;
T→C within codon 10 of the gene encoding TGFβ;
760 C/G in the gene encoding SOD3;
−1296 T/C within the promoter of the gene encoding TIMP3;
the S mutation in the gene encoding α1-antitrypsin; and
one or more polymorphisms that are in linkage disequilibrium with one or more of these further polymorphisms.

4. The method according to claim 1, wherein said method comprises the analysis of one or more epidemiological risk factors.

5. A method of determining a subject's risk of developing chronic obstructive pulmonary disease, emphysema, or both chronic obstructive pulmonary disease and emphysema, the method comprising:
analysing a sample from said subject for the presence or absence of one or more polymorphisms selected from the group consisting of:
rs10115703 G/A polymorphism in the gene encoding Cer 1;
rs13181 G/T polymorphism in the gene encoding XPD;
rs1799930 G/A polymorphism in the gene encoding NAT2;
rs2031920 C/T polymorphism in the gene encoding CYP2E1;
rs4073 T/A polymorphism in the gene encoding IL-8;
rs763110 C/T polymorphism in the gene encoding FasL;
rs16969968 G/A polymorphism in the gene encoding α5-nAChR;
rs1051730 C/T polymorphism in the gene encoding α5-nAChR; and
one or more polymorphisms in linkage disequilibrium with one or more of these polymorphisms;
wherein the presence or absence of one or more of said polymorphisms is indicative of the subject's risk of developing COPD, emphysema, or both COPD and emphysema.

6. The method of claim 5, additionally comprising:
analysing the sample from said subject for the presence or absence of one or more further polymorphisms selected from the group consisting of:
the rs4934 G/A polymorphism in the gene encoding α1 anti-chymotrypsin;
the rs1489759 A/G polymorphism in the gene encoding HHIP; and
the rs2202507 A/C polymorphism in the gene encoding GYPA.

7. The method according to claim 5, wherein the method comprises the analysis of one or more epidemiological risk factors.

8. One or more nucleotide probes or primers for use in the method of claim 3, wherein the one or more nucleotide probes and/or primers span, or are able to be used to span, the polymorphic regions of the genes in which the polymorphism to be analysed is present.

9. The one or more nucleotide probes or primers of claim 8, wherein the probe or primer spans or is able to be used to span one or more of the polymorphisms selected from the group consisting of:
the rs10115703 G/A polymorphism in the gene encoding Cer 1;
the rs13181 G/T polymorphism in the gene encoding XPD;
the rs1799930 G/A polymorphism in the gene encoding NAT2;
the rs2031920 C/T polymorphism in the gene encoding CYP2E1;
the rs4073 T/A polymorphism in the gene encoding IL-8;
the rs763110 C/T polymorphism in the gene encoding FasL;
the rs16969968 G/A polymorphism in the gene encoding α5-nAChR; and
the rs1051730 C/T polymorphism in the gene encoding α5-nAChR.

10. A probe or primer according to claim 9, comprising the sequence of any one of SEQ. ID. NO. 1 to 38.

11. A pair of primers comprising two primers as claimed in claim 8.

12. A nucleic acid microarray for use in the methods according to claim 3, which microarray comprises a substrate presenting nucleic acid sequences capable of hybridizing to nucleic acid sequences which encode one or more of the polymorphisms selected from the group defined in claim 3 or sequences complimentary thereto.

13. A method treating a subject having an increased risk of developing COPD, emphysema, or both COPD and emphysema comprising the step of:
replicating, in said subject, genotypically or phenotypically, the presence and/or functional effect of a protective polymorphism selected from the group consisting of:
the G allele at the rs13181 polymorphism in the gene encoding XPD;
the GG genotype at the rs13181 polymorphism in the gene encoding XPD;
the T allele at the rs763110 polymorphism in the gene encoding FasL;
the TT genotype at the rs763110 polymorphism in the gene encoding FasL;
the G allele at the rs1489759 polymorphism in the gene encoding HHIP;
the GG genotype at the rs1489759 polymorphism in the gene encoding HHIP;
the C allele at the rs2202507 polymorphism in the gene encoding GYPA; and
the CC genotype at the rs2202507 polymorphism in the gene encoding GYPA.

14. (canceled)

15. An antibody microarray which comprises a substrate presenting antibodies capable of binding to a product of expression of a gene the expression of which is upregulated or downregulated when associated with a polymorphism selected from the group defined in claim 2.

16. A method for screening for compounds that modulate the expression and/or activity of a gene, the expression of which is upregulated or downregulated when associated with a polymorphism selected from the group defined in claim 2, said method comprising the steps of:
contacting a candidate compound with a cell comprising a polymorphism selected from the group defined in claim 2 which has been determined to be associated with the upregulation or downregulation of expression of a gene; and
measuring the expression of said gene following contact with said candidate compound,
wherein a change in the level of expression after the contacting step as compared to before the contacting step is indicative of the ability of the compound to modulate the expression and/or activity of said gene.

17. The method according to claim 16, wherein said cell is a human lung cell which has been pre-screened to confirm the presence of said polymorphism.

18. The method according to claim 17, wherein said cell comprises a susceptibility polymorphism associated with downregulation of expression of said gene and said screening is for candidate compounds which upregulate expression of said gene.

19. The method according to claim 17, wherein said cell comprises a susceptibility polymorphism associated with downregulation of expression of said gene and said screening is for candidate compounds which upregulate expression of said gene.

20. The method according to claim 17, wherein said cell comprises a protective polymorphism associated with upregulation of expression of said gene and said screening is for candidate compounds which further upregulate expression of said gene.

21.-31. (canceled)
